(19) United States
(12) Patent Application Publication
(10) Pub. No.: US 2021/0317071 A1
(43) Pub. Date: Oct. 14, 2021

(54) PH-RESPONSIVE LIPIDS

(71) Applicant: University of KwaZulu-Natal, Durban (ZA)

(72) Inventors: Thirumala Govender, Durban (ZA); Mahantesh Jadhav, Durban (ZA); Rahul Kalhapure, Durban (ZA); Chunderika Mocktar, Durban (ZA); Sanjeev Kumar Rambharose, Durban (ZA)

(21) Appl. No.: 16/326,599

(22) PCT Filed: Aug. 18, 2017
(86) PCT No.: PCT/IB2017/055010

§ 371 Date: Feb. 19, 2019

(30) Foreign Application Priority Data
Aug. 18, 2016 (GB) 1614120.2

(51) Int. Cl. C07C 229/16; C07C 229/12; C07C 227/16; A61K 9/127

(52) U.S. Cl. CPC C07C 229/16; A61K 9/127; C07C 227/16; C07C 229/12

(57) ABSTRACT

The invention provides for a synthesised ester intermediate of formula 1. Formula 1 Wherein and wherein R may be a saturated or unsaturated fatty acid (C12-C20).

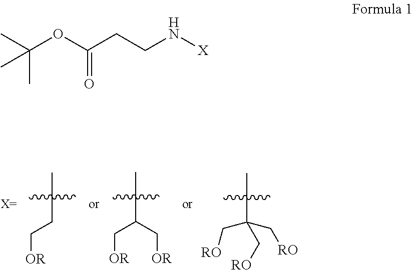

This invention relates to novel pH-responsive lipids and their ester intermediates, their synthesis and use.

### BACKGROUND TO THE INVENTION

There is a growing demand in the pharmaceutical industry for pH-responsive lipids due to their use in formulating pH-responsive drug delivery system (PSDDS). These drug delivery systems ensure the delivery of a drug at a specific site as per the pathological need of the disease being treated, resulting in improved therapeutic efficacy. Diseases wherein PSDDS are employed include bacterial infections, asthma, peptic ulcers and cancer. pH-responsive lipids have gained renewed interest as lipidic excipients for the development of targeted drug delivery systems, such as liposomes, vesicles composed of amphipathic lipids arranged in spherical bilayers. Liposomes may be used to encapsulate various drugs, by trapping hydrophilic drugs in the aqueous interior or between bilayers, or by trapping hydrophobic compounds within the bilayer (Med. Chem. Comm. 2014, 5, 1602-1618). Conventional liposomes are mainly composed of natural or synthetic phospholipids and cholesterol (Int. J. Pharm. 2010, 387, 187-198). Lipids are also used as penetration enhancers, emulsifying and solubilizing agents in pharmaceutical formulations. Following the development of an increasing number of insoluble drugs, and an emphasis on precise performance and alternative routes of administration, there is a need for new lipidic excipients, so as to provide a greater choice for the development of novel, biocompatible, non-irritating and cost-effective lipidic nano and/or micro drug delivery systems.

Approved liposomal formulations in the market include first generation conventional liposomes (Myocet/Daunoxome) and their PEGylated forms (Doxil/Lipo-Dox) for extended circulation. Second generation liposomal drug delivery system endeavours include broad therapeutic applications from dual drug loaded liposomes (CPX-1/CPX-351) to stimuli response liposomes (ThermoDox). The current focus of drug delivery research is to develop universal responsive drug carriers for targeted delivery.

The concept of pH-responsive liposomes, was first introduced by Yatvin et al (Science, 1980, 21012, 1253-1255), where it was proposed that pH-responsive liposomes could be used as drug carriers, releasing their payload at the desired site, where the pH is lower than physiological pH (7.4). Since then, further research has been conducted on the design and synthesis of semisynthetic and synthetic lipids with desired biophysical properties that can be exploited for the development of pH-responsive liposomes to promote efficient drug delivery at targeted site while retaining low cytotoxicity and immunogenicity. The sensitivity of liposomes can be precisely engineered by incorporating lipids with physicochemical behaviour that is regulated by surrounding pH. While lipid tails primarily modulate bilayer phase behaviour, it is the head group that determines the bilayer surface chemistry.

Lipids having an environmentally sensitive head group are desirable because the net charge of these molecules can be cationic, neutral or anionic as dictated by the pH of the surrounding environment. Lipids with an anionic head group at physiological pH can be transformed into neutral or cationic phase upon a change in an environmental pH, and will deliver content at the desired site with low pH. Anionic lipids also facilitate the encapsulation of many basic drugs such as antimicrobial peptides, peptide antibiotics among other and promotes the delivery at targeted site.

Over the last four decades, numerous anionic and cationic lipids have been synthesized and used as pH-responsive materials for preparation of liposomes capable of delivering drug at the desired site. These lipids include fatty acids, cholesterol hemisuccinate (CHEMS), phosphatidic acid phosphatidylethanolamine (PE), distearyl-phosphatidylethanolamine (DSPE), trans-2-cyclohexanol, mono stearoyl morpholine derivatives, and cyclen-based cationic lipids with histidine moiety. Amino acid based pH-responsive or zwitterionic lipids have been found to improve the lipid membrane interaction and intracellular delivery of drugs, proteins, and RNA.

Naturally occurring pH-responsive, or zwitterionic lipids (e.g. phosphatidylcholine (PC) and phosphatidylethanolamine (PE)) and their derivatives have been used in the formulation of pH-responsive liposomes. More recently, phospholipid based formulations, such as Doxile®, Cleviprex®, Valium® and Silybin Phytosome™, have been used in clinics. However, despite excellent biocompatibility and wide applications in drug delivery systems, use of phospholipids is limited, due to the fact that naturally occurring lipids are impure and their purification is difficult and synthetic phospholipids are very expensive to produce.

Besides the use of lipids in nano drug delivery systems such as liposomes, nanoemulsion, and solid lipid nanoparticles, they are also in demand for their use as chemical permeation enhancers (CPEs) to systemically deliver bioactives. Among all the types of lipids, fatty acids containing long hydrocarbon chains such as oleic acid (Drug Deliv. 2008, 15, 303-309) and their derivatives have shown promising results as CPEs (J. Mater. Chem. B. 2015, 3, 6662-6675; Drug Dev. Ind. Pharm. 2014, 40, 657-668). It has been reported that only 1 in 100,000 molecules represents a CPE (Proc. Natl. Acad. Sci. USA, 2005, 102, 4688-4693). New CPEs are always in demand by drug delivery scientists because the delivery of bioactives using CPEs is an attractive alternative for conventional delivery routes.

It is an object of this invention to provide bio-safe pH-responsive lipids and their resultant responsive liposomes for targeted nano drug delivery application as well as ester intermediates of the synthesized bio-safe pH-responsive lipids as transdermal permeation enhancers which, at least partially, alleviates some of the above mentioned problems.

### SUMMARY OF THE INVENTION

In accordance with this invention, there is provided a synthesised ester intermediate of formula 1.

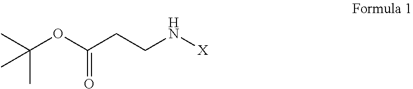

Wherein

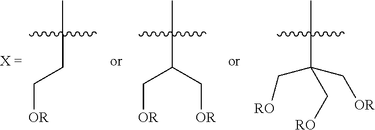

and wherein R may be a saturated or unsaturated fatty acid (C12-C20).

The synthesised ester intermediate may comprise 1 or more fatty acid chains, and preferably comprises 1 to 3 saturated or unsaturated fatty acid chains.

R preferably comprises any of C₁₈H₃₆O₂ (stearic acid), C₁₈H₃₄O₂ (oleic acid), C₁₈H₃₂O₂ (linoleic acid) or C₁₈H₃₀O₂ (linolenic acid).

In one embodiment of the invention, the synthesized ester intermediate of formula 1 comprises a hydrophilic head group, functionalized with beta-amino propionic acid (beta alanine) tert butyl ester and connected to 1, 2 or 3 saturate or unsaturated fatty acid chains (hydrophobic tails) through an acid-labile ester bond or linker.

The linker preferably comprises 2-aminoethanol or ethanolamine (HO(CH₂)₂NH₂), 2-amino-1,3-propanediol or serinol ((HOCH₂)₂CHNH₂), and 2-amino-2-(hydroxymethyl)propane-1,3-diol (trizma or Trisaminomethane) ((HOCH₂)₃CNH₂).

The synthesised ester intermediate of formula 1 may comprise one or more or the following: 2-((3-(tert-butoxy)-3-oxopropyl)amino)ethyl stearate (MSAPE); 2-((3-(tert-butoxy)-3-oxopropyl)amino)ethyl oleate (MOAPE); 2-((3-(tert-butoxy)-3-oxopropyl)amino)ethyl (9Z,12Z)-octadeca-9,12-dienoate (MLAPE); 2-((3-(tert-butoxy)-3-oxopropyl)amino)ethyl (9Z,12Z,15Z)-octadeca-9,12,15-trienoate (MLLAPE); 2-((3-(tert-butoxy)-3-oxopropyl)amino)propane-1,3-diyl distearate (DSAPE); 2-((3-(tert-butoxy)-3-oxopropyl)amino)propane-1,3-diyl dioleate (DOAPE); 2-((3-(tert-butoxy)-3-oxopropyl)amino)propane-1,3-diyl (9Z,9′Z,12Z,12′Z)-bis(octadeca-9,12-dienoate) (DLAPE); 2-((3-(tert-butoxy)-3-oxopropyl)amino)propane-1,3-diyl (9Z,9′Z,12Z,12′Z,15Z,15′Z)-bis(octadeca-9,12,15-trienoate) (DLLAPE); 2-((3-(tert-butoxy)-3-oxopropyl)amino)-2-((stearoyloxy)methyl) propane-1,3-diyl distearate, (TSAPE); 2-((3-(tert-butoxy)-3-oxopropyl)amino)-2-((((Z)-octadec-9-enoyl)oxy)methyl)propane-1,3-diyl(9Z,9′Z)-bis(octadec-9-enoate) (TOAPE); 2-((3-(tert-butoxy)-3-oxopropyl)amino)-2-((((9Z,12Z)-octadeca-9,12-dienoyl)oxy)methyl)propane-1,3-diyl (9Z,9′Z,12Z,12′Z)-bis(octadeca-9,12-dienoate); 2-((3-(tert-butoxy)-3-oxopropyl)amino)-2-((((9Z,12Z,15Z)-octa-dec-9,12,15-trienoyl)oxy)methyl)propane-1,3-diyl(9Z,9′Z,12Z,12′Z,15Z,15′Z)-bis(octadeca-9,12,15-trienoate) (TLLAPE).

The terminal ester group of the synthesised ester intermediate of formula 1 may be hydrolysed to create a pH-responsive lipid of formula 2a, 2b or 2c.

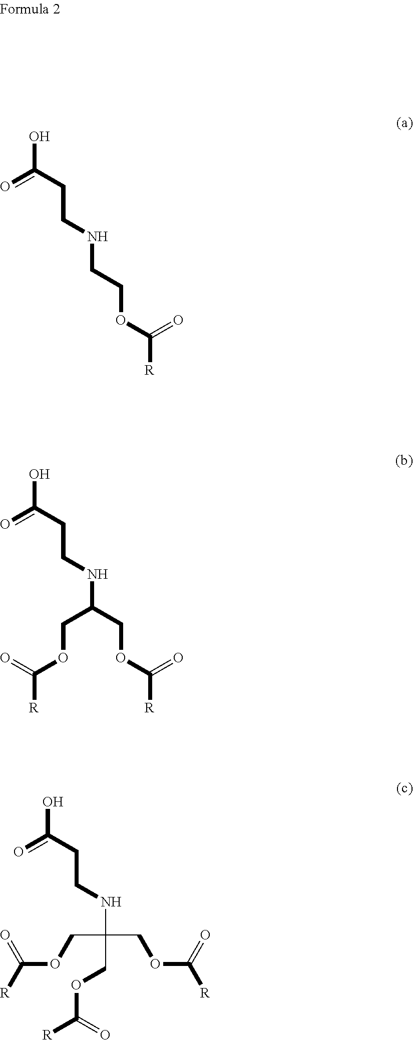

The invention also extends to a synthesised pH-responsive lipid of formula 2 (a, b or c) where R may be a saturated or unsaturated fatty acid chain (C12-C20) and is preferably any of C₁₈H₃₆O₂ (stearic acid), C₁₈H₃₄O₂ (oleic acid), C₁H₃₂O₂ (linoleic acid) or C₁₈H₃₀O₂ (linolenic acid):

The synthesised pH-responsive lipid of formula 2 preferably comprises a hydrophilic head group, functionalized with beta-amino propionic acid (beta alanine) and connected to 1, 2 or 3 fatty acid chains (hydrophobic tails) through an acid-labile ester bond.

There is further provided for the synthesised pH-responsive lipid of formula 2 to comprise any of the following:

2(a): 3-((2-(stearoyloxy)ethyl)amino)propanoic acid (MSAPA); 3-((2-(oleoyloxy)ethyl)amino)propanoic acid (MOAPA); 3-((2-(((9Z,12Z)-octadeca-9,12-dienoyl)oxy)ethyl)amino)propanoic acid (MLAPA); 3-((2-(((9Z,12Z,15Z)-octadeca-9,12,15-trienoyl)oxy)ethyl)amino)propanoic acid (MLLAPA);
2(b): 3-((1,3-bis(stearoyloxy)propan-2-yl)amino)propanoic acid (DSAPA); 3-((1,3-bis(oleoyloxy)propan-2-yl)amino)propanoic acid (DOAPA); 3-((1,3-bis(((9Z,12Z)-octadeca-9,12-dienoyl)oxy)propan-2-yl)amino)propanoic acid (DLAPA); 3-((1,3-bis(((9Z,12Z,15Z)-octadeca-9,12,15-trienoyl)oxy)propan-2-yl)amino)propanoic acid (DLLAPA); or 2(c): 3-((1,3-bis(stearoyloxy)-2-((stearoyloxy)methyl)propan-2-yl)amino) propanoic acid (TSAPA); or 3-((1,3-bis(((Z)-octadec-9-enoyl)oxy)-2-((((Z)-octadec-9-enoyl)oxy)methyl)propan-2-yl) amino)propanoic acid (TOAPA); or 3-((1,3-bis(((9Z,12Z)-octadeca-9,12-dienoyl)oxy)-2-((((9Z,12Z)-octadeca-9,12-dienoyl)oxy)methyl)propan-2-yl)amino) propanoic acid (TLAPA) or 3-((1,3-bis(((9Z,12Z,15Z)-octadeca-9,12,15-trienoyl)oxy)-2-((((9Z,12Z,15Z)-octadeca-9,12,15-trienoyl)oxy)methyl)propan-2-yl)amino) propanoic acid (TLLAPA).

The invention further extends to a method of synthesising pH-responsive lipids which contain specifically a secondary amine group by selective mono Michael addition reaction in between amino group of ethanolamine or serinol or trizma with tert-butyl acrylate at specific reaction conditions [compound 3; tert-butyl 3-((2-hydroxyethyl)amino)propanoate (scheme 1), compound 6; tert-butyl 3-((1,3-dihydroxypropan-2-yl)amino)propanoate (scheme 2), compound 9; tert-butyl 3-((1, 3-dihydroxy-2-(hydroxymethyl)propan-2-yl)amino)propanoate (scheme 3)].

The invention further extends to nanosystems comprising the pH-responsive lipid and/or a liposome containing them. In this embodiment of the invention, the liposome may comprise a pH-responsive lipid of the invention and one or more additional lipid compounds.

The liposome may comprise between 5 and 40 w/w % of said pH-responsive lipid of formula I and preferably between 5 and 20%.

The additional lipid compound may be any of cholesterol, phosphatidylcholine (PC) phosphatidyl ethanolamine, ceramide, sphingolipid, tetraether lipid, diacylglycerol, phosphatidylserine, phosphatidic acid or CHEMS.

Preferably, the liposome comprises a pH-responsive lipid of the invention and two additional lipid compounds, and the ratio of pH-responsive lipid, phosphatidylcholine and cholesterol may be 1:3:1 (w/w/w).

The liposome may have an average size in between 80 to 600 nm.

The invention encompasses the design and synthesis of novel pH-responsive lipids for the delivery of bioactive pharmaceutical agents, including but not limited to small molecules, lipids, nucleosides, nucleotides, nucleic acids, polynucleotides, oligonucleotides, antibodies, toxins, negatively charged polymers and other polymers, for example proteins, peptides, hormones, carbohydrates, or polyamines across cellular membranes.

Accordingly, the liposome may additionally comprise a medically active substance such as drugs molecules, peptides nucleosides, nucleotides, nucleic acids, polynucleotides, oligonucleotides, antibodies, and toxins.

Further embodiments of the invention may include a composition comprising at least one pH-responsive lipid of the invention and a pharmaceutical substance. The pharmaceutical substance may include cholesterol and/or phosphatidylcholine (PC) phosphatidyl ethanolamine, ceramide, sphingolipid, tetraether lipid, or diacylglycerol, phosphatidylserine, phosphatidic acid or CHEMS.

The invention still further extends to a pharmaceutical composition comprising at least one pH-responsive lipid and a pharmaceutically tolerable carrier, as well as to the use of the pH-responsive liposomes as pH-responsive nano drug delivery system for site-specific drug delivery.

The present invention is further extended to the use of the ester intermediates of formula 1 as chemical permeation enhancers for drug delivery applications. The invention encompasses the design and synthesis of novel lipidic esters for the transdermal delivery of bioactive pharmaceutical agents, including but not limited to small molecules, lipids, nucleosides, nucleotides, nucleic acids, polynucleotides, oligonucleotides, antibodies, toxins, negatively charged polymers and other polymers, for example proteins, peptides, hormones, carbohydrates, or polyamines.

### BRIEF DESCRIPTION OF THE ILLUSTRATIONS

A preferred embodiment of the invention is described below by way of example only and with reference to the following figures in which;

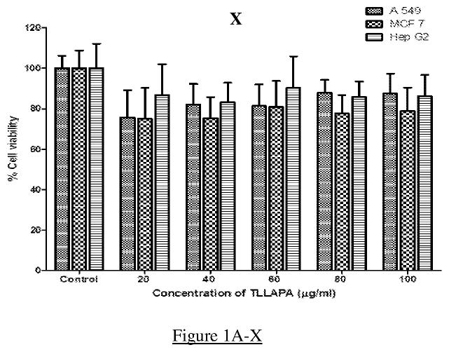

FIG. 1 is a graphical representation (A to X) of the cytotoxicity of all the synthesized lipids of the invention at various concentrations against (I) human liver hepatocellular carcinoma (HepG2), (II) human breast adenocarcinoma (MCF 7) and (III) human cervix adenocarcinoma (HeLa)) cell lines;

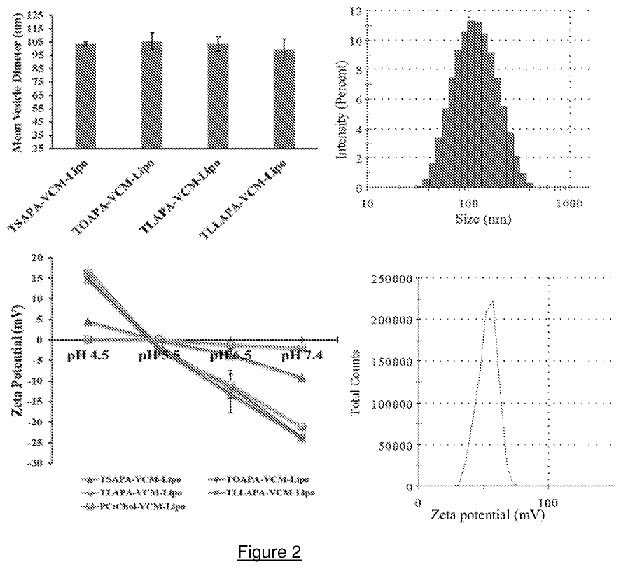

FIG. 2 is a graphical representation of the pH-responsive Liposomes' (pH-responsive lipid containing lipids of formula 2c) particle size, zeta potential as a function of pH. Data is presented as the mean±SD (n=3);

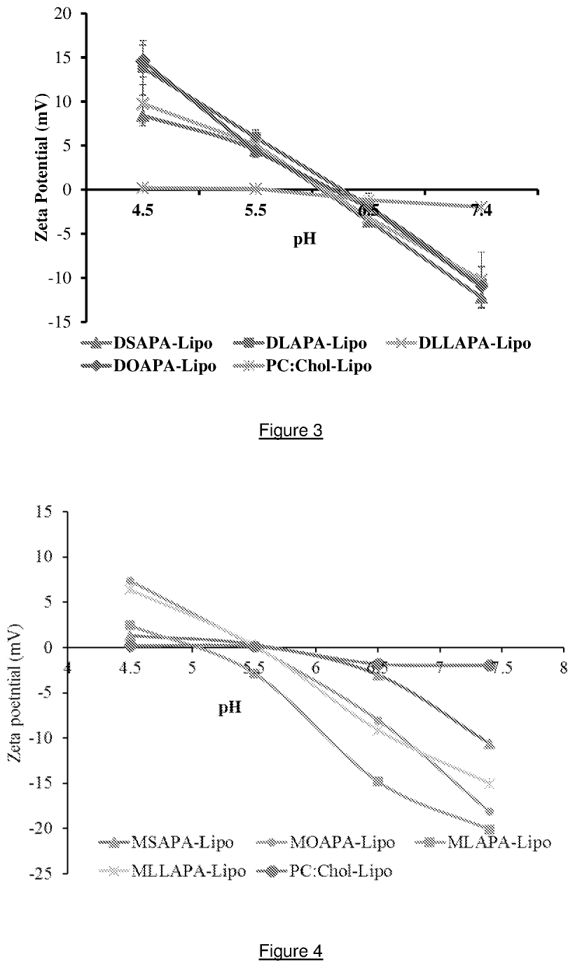

FIG. 3 is a graphical representation of the pH-responsive Liposomes' (pH-responsive lipid containing lipids of formula 2b) zeta potential as a function of pH. Data is presented as the mean±SD (n=3);

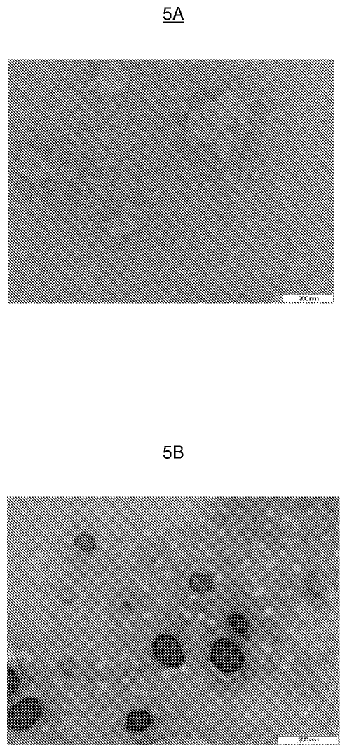

FIG. 4 is a graphical representation of the pH-responsive Liposomes' (pH-responsive lipid containing lipids of formula 2a) zeta potential as a function of pH. Data is presented as the mean±SD (n=3);

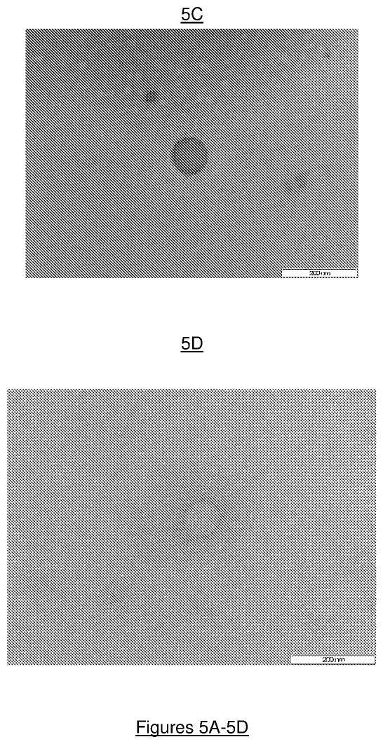

FIG. 5a-d are representative Transmission Electron Microscopic images of VCM loaded liposome containing pH-responsive lipid;

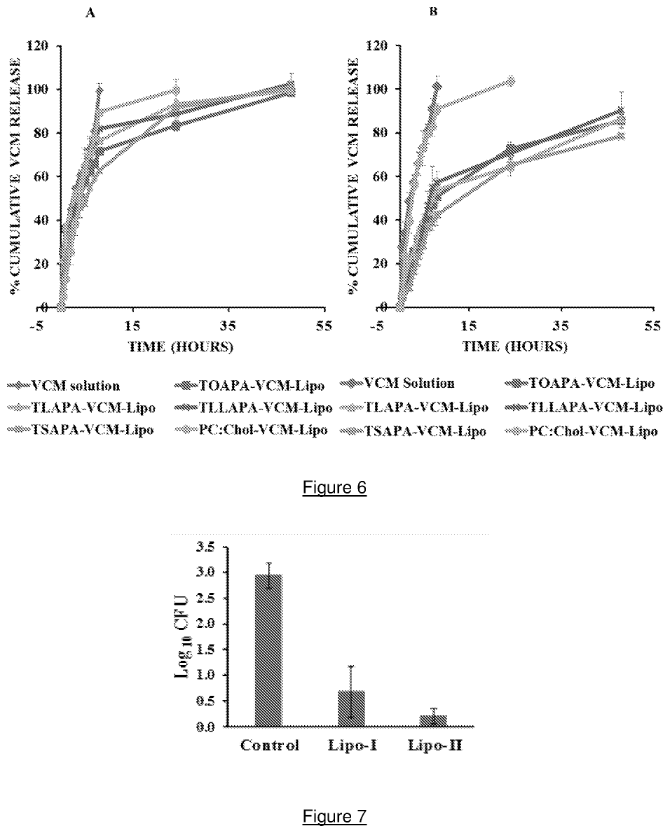

FIG. 6 is a graphical representation of in-vitro VCM release from liposomes as a function of pH. Data is presented as the mean±SD (n=3);

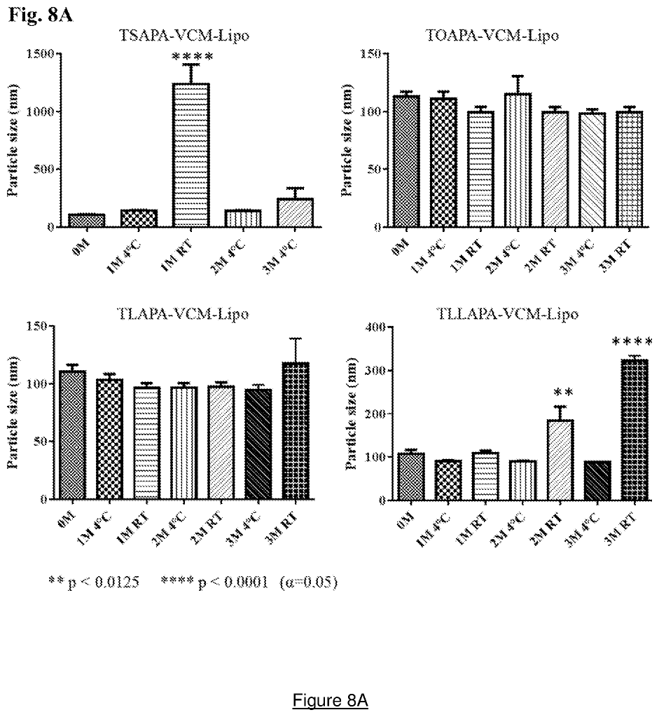

FIG. 7 is a graphical representation of total colony forming units (CFU) in mouse skin infections treated with VCM loaded pH-responsive liposomes of the invention.

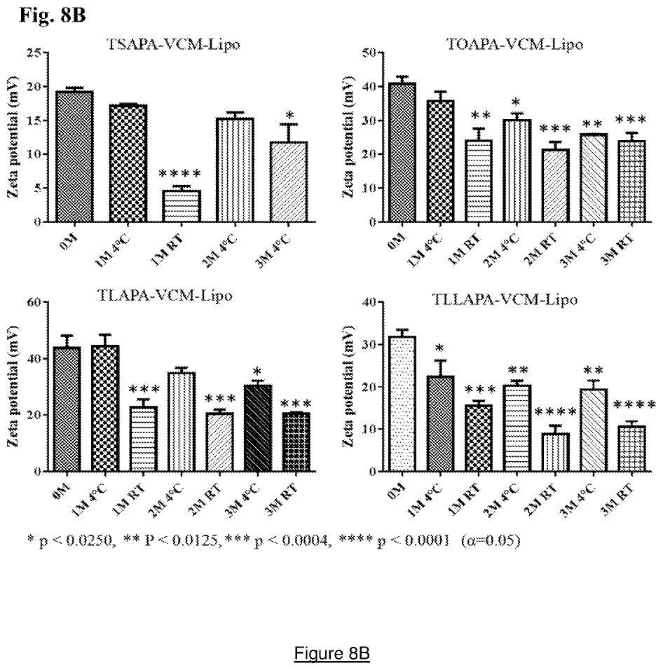

FIGS. 8A and 8B are a graphical representation of the storage stability of the liposomal formulations (TSAPA-VCM-Lipo, TOAPA-VCM-Lipo, TLAPA-VCM-Lipo, and TLLAPA-VCM-Lipo) over three months at 4° C. and RT. The storage stability indicators (8A) MVD and (8B) ZP; data is presented as the mean±SD (n=3).

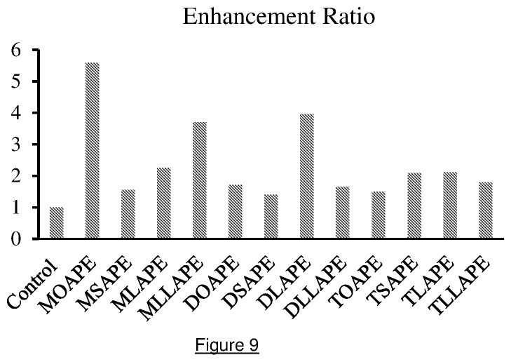

FIG. 9 is a graphical representation of enhancement ratio of Tenofivir (TNF) studied at 1% (w/w) concentration of ester intermediates of formula 1. Data is presented as the mean±SD (n=3).

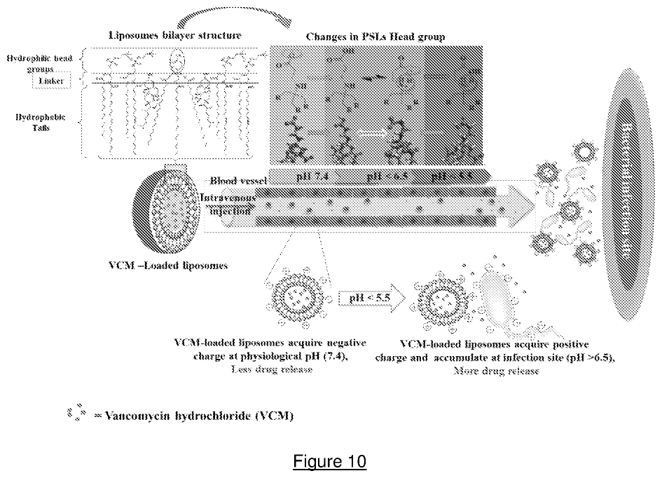

FIG. 10 is a graphical representation of a plausible mechanism by which the pH-responsive liposomes operate as a targeted drug delivery system.

### DETAILED DESCRIPTION OF THE INVENTION

Referring to FIGS. 1 to 10 an exemplary model of the synthesis and characterisation of the pH-responsive lipids of the invention, their use in the formulation of pH-responsive liposomes, and the characterisation of the resultant pH-responsive liposomes and their use in providing drug delivery systems is described below.

1. Synthesis and Characterization of DH-Responsive Lipids

In general, the lipids of the invention are prepared by art-recognized reactions. A number of exemplary synthetic routes are set forth herein for the purposes of illustration, however, the scope of this illustration is not intended to be limiting.

The design, synthesis, and characterization of the novel class of pH-responsive Lipids is described below with reference to Scheme 1-3.

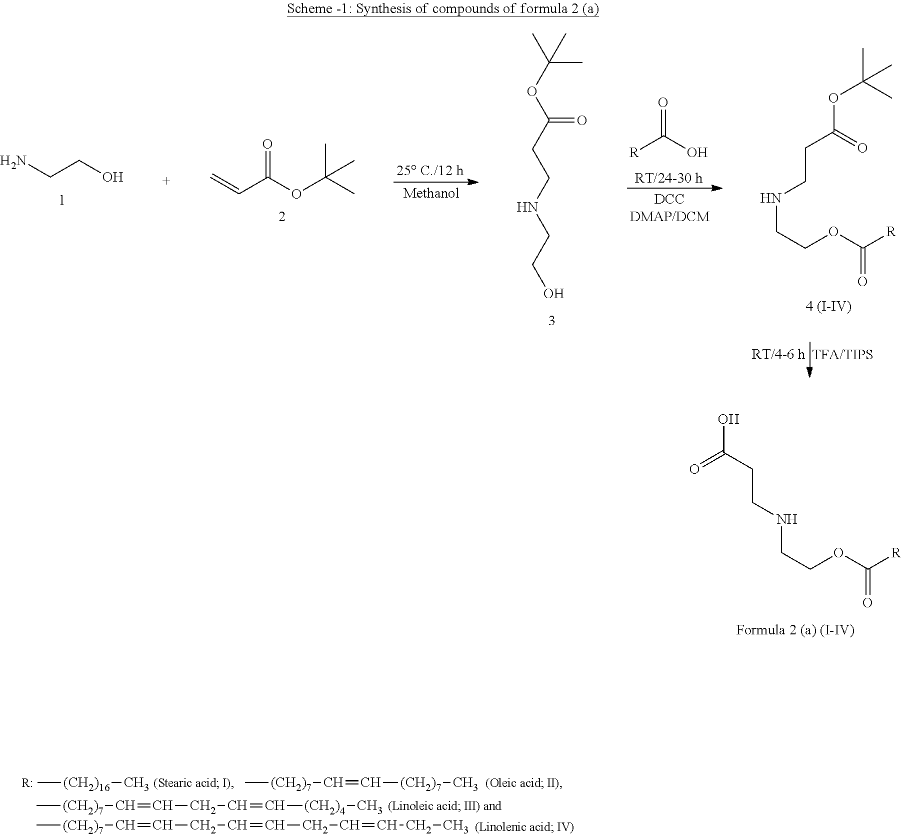

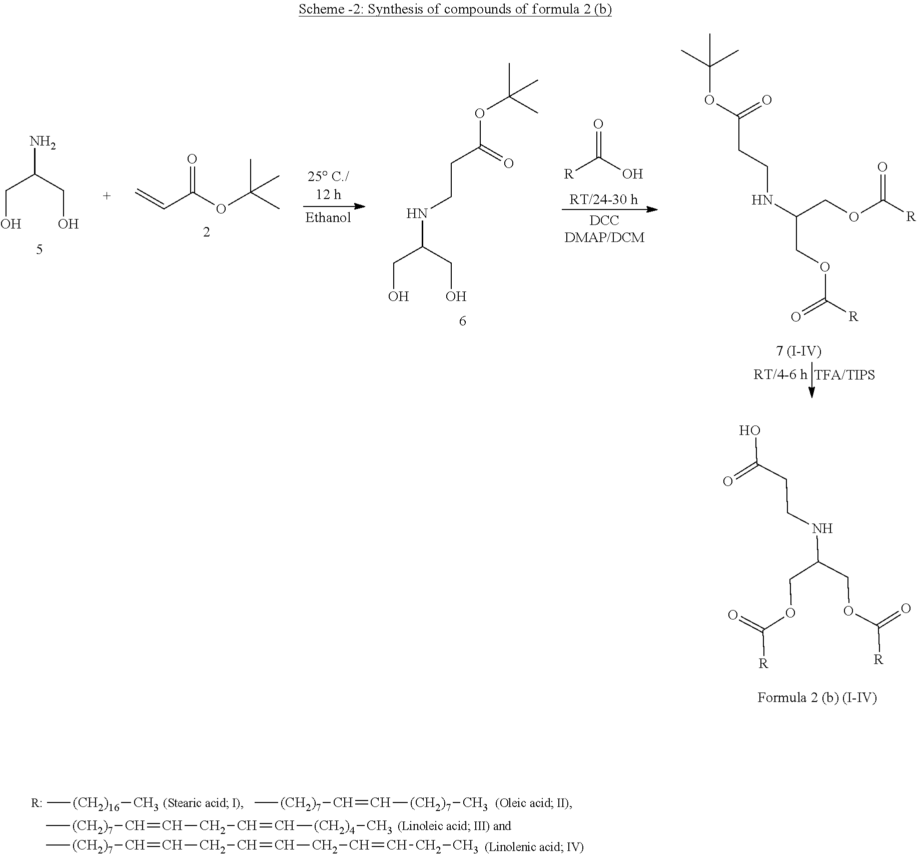

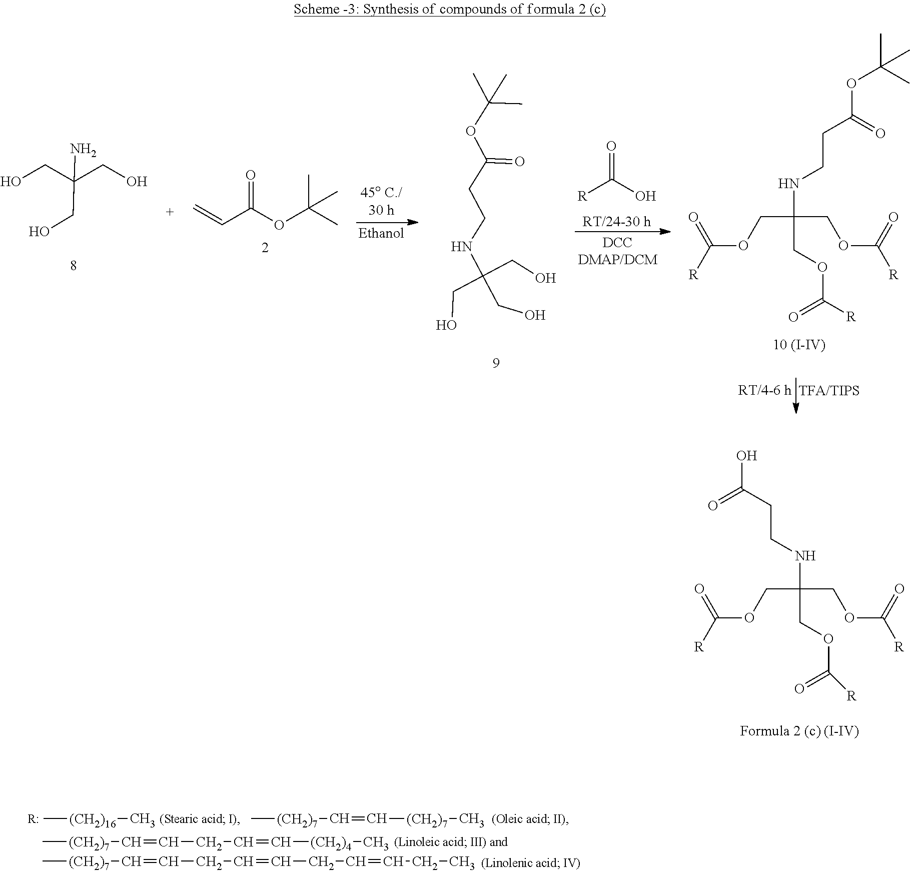

### Schemes 1-3

The novel class of synthesized pH-responsive lipids of formula 2 consist of a hydrophilic head group, functionalized with beta-amino propionic acid (beta alanine) and connected to one to three fatty acid chains (hydrophobic tails) through acid-labile ester bond. The pH-responsive lipids were engineered and synthesized from biocompatible and biodegradable materials. pH-responsive lipids are made up of a bio-safe linker part, ethanolamine (2-aminoethanol) (compound 1 of scheme 1); serinol (2-amino-1,3-propanediol) (compound 5 of scheme 2); or Trizma (2-amino-2-(hydroxymethyl)propane-1,3-diol) (compound 8 of scheme 3), and fatty acids (stearic, oleic, linoleic and linolenic acid (R)). The secondary amine in the resultant pH-responsive lipids can be protonated at acidic pH and it is capable of forming zwitterion due to the adjacent carboxylic acid group. Thus the beta-amino alanine head group is responsible for pH-dependent ionization and adaptation of inter and/or intra molecular interactions through H-bonding, causing a slight conformational flip in the hydrophobic tails.

As depicted in schemes 1-3 above, a three-step synthetic route was employed to synthesize the pH-responsive lipids with different alkyl chains. Key intermediates, tert butyl 3-((2-hydroxyethyl)amino)propanoate (compound 3; scheme 1); tert-butyl 3-((1,3-dihydroxypropan-2-yl)amino)propanoate (compound 6; scheme 2); tert-butyl 3-((1,3-dihydroxy-2-(hydroxymethyl)propan-2-yl)amino)propanoate (compound 9; scheme 3) were obtained quantitatively via single Michael addition reaction between the amine (compounds 1, 5 or 8) and tert-butyl acrylate (compound 2).

This method has previously been reported for the synthesis of esters containing tertiary amino alcohol derivative via double Michael additions with amino mono-alcohol (3-amino-1-propanol) and methyl acrylate or tert-butyl acrylate, and amino tri-alcohols (Trizma) and methyl acrylate. However, in our case single Michael addition reaction between trizma and tert butyl acrylate was observed. Under the same reaction conditions, the reaction was not specifically mono-addition when trizma was replaced with serinol or 2-aminoethanol. We specifically obtained mono addition products for serinol and 2-aminoethanol by controlling reaction conditions and equivalent of tert butyl acrylate used.

The intermediate (compound 3, 6 or 9) was coupled to stearic acid (SA), oleic acid (OA), Linoleic acid (LA) and Linolenic acid (LLA) by Steglich esterification using N,N′-di cyclohexyl carbodiimide (DCC) as a coupling reagent to obtain mono- (scheme-1), di-(scheme-2) or tri-(scheme-3) substituted ester derivatives (4, 7 or 10; formula 1) with good yield (70-83%).

Finally, hydrolysis of the terminal ester group of formula 1 was achieved under acidic conditions to yield final pH-responsive lipids (formula 2a-c). The structures of all the synthesized intermediates and final pH-responsive lipids were confirmed by FTIR, NMR (¹H and ¹³C) and HRMS analysis.

The synthesised ester intermediates of formula 1 were named with the following acronyms:

### Mono-Stearoyl Amino Propionic Acid Tert-Butyl Ester (MSAPE)

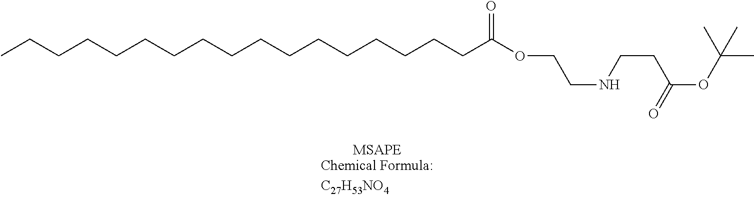 [SMILES: CCCCCCCCCCCCCCCCCC(=O)OCCNCCC(=O)OC(C)(C)C]

### Mono-Oleoy Amino Propionic Acid Tert-Butyl Ester (MOAPE)

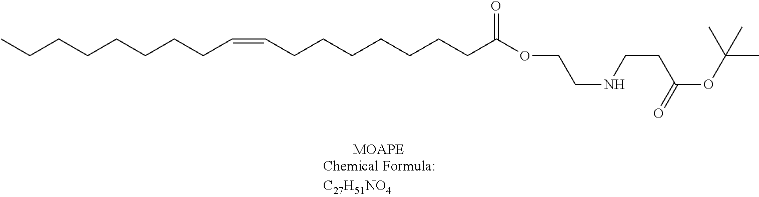 [SMILES: CCCCCCCC/C=C\CCCCCCCC(=O)OCCNCCC(=O)OC(C)(C)C]

### Mono-Linolenoyl Amino Propionic Acid Tert-Butyl Ester (MLAPE)

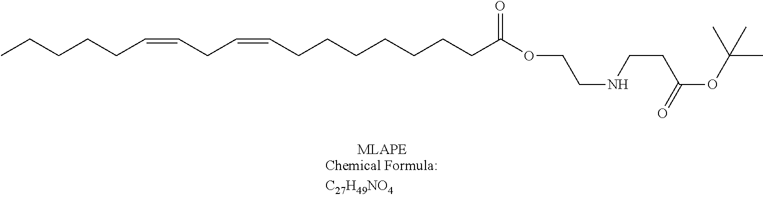 [SMILES: CCCCC/C=C\C/C=C\CCCCCCCC(=O)OCCNCCC(=O)OC(C)(C)C]

### Mono-LinoLenoyl Amino Propionic Acid Tert-Butyl Ester (MLLAPE)

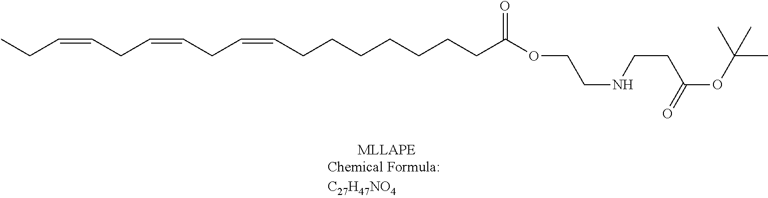 [SMILES: CC/C=C\C/C=C\C/C=C\CCCCCCCC(=O)OCCNCCC(=O)OC(C)(C)C]

### Di-Stearoyl Amino Propionic Acid Tert-Butyl Ester (DSAPE)

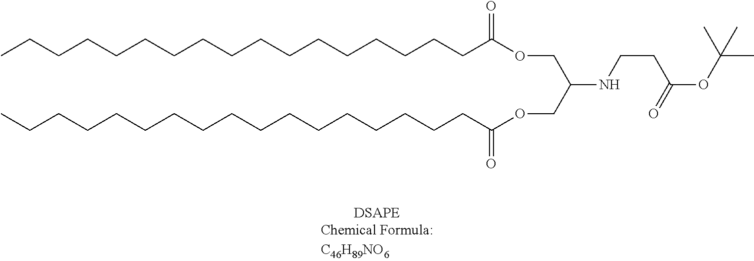 [SMILES: CCCCCCCCCCCCCCCCCC(=O)OCC(COC(=O)CCCCCCCCCCCCCCCCC)NCCC(=O)OC(C)(C)C]

### Di-Oleoyl Amino Propionic Acid Tert-Butyl Ester (DOAPE)

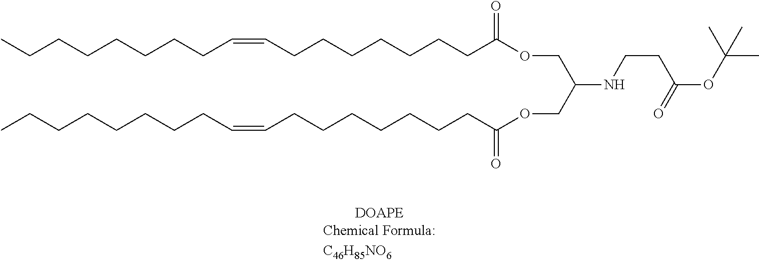 [SMILES: CCCCCCCC/C=C\CCCCCCCC(=O)OCC(COC(=O)CCCCCCC/C=C\CCCCCCCC)NCCC(=O)OC(C)(C)C]

### Di-Linoleoyl Amino Propionic Acid Tert-Butyl Ester (DLAPE)

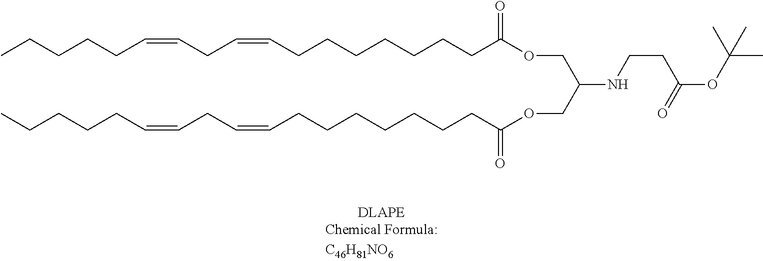 [SMILES: CCCCC/C=C\C/C=C\CCCCCCCC(=O)OCC(COC(=O)CCCCCCC/C=C\C/C=C\CCCCC)NCCC(=O)OC(C)(C)C]

### Di-LinoLenoyl Amino Propionic Acid Tert-Butyl Ester (DLLAPE)

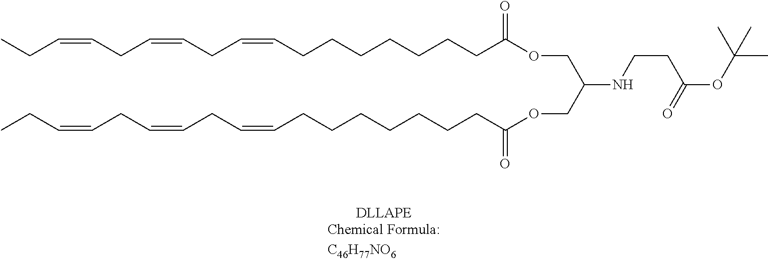 [SMILES: CC/C=C\C/C=C\C/C=C\CCCCCCCC(=O)OCC(COC(=O)CCCCCCC/C=C\C/C=C\C/C=C\CC)NCCC(=O)OC(C)(C)C]

### Tri-Stearoyl Amino Propionic Acid Tert-Butyl Ester (TSAPE)

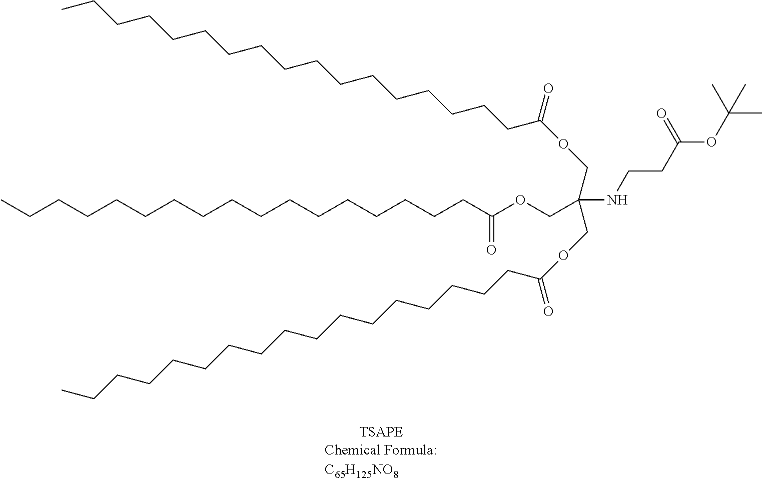 [SMILES: CCCCCCCCCCCCCCCCCC(=O)OCC(COC(=O)CCCCCCCCCCCCCCCCC)(COC(=O)CCCCCCCCCCCCCCCCC)NCCC(=O)OC(C)(C)C]

### Tri-Oleoyl Amino Propionic Acid Tert-Butyl Ester (TOAPE)

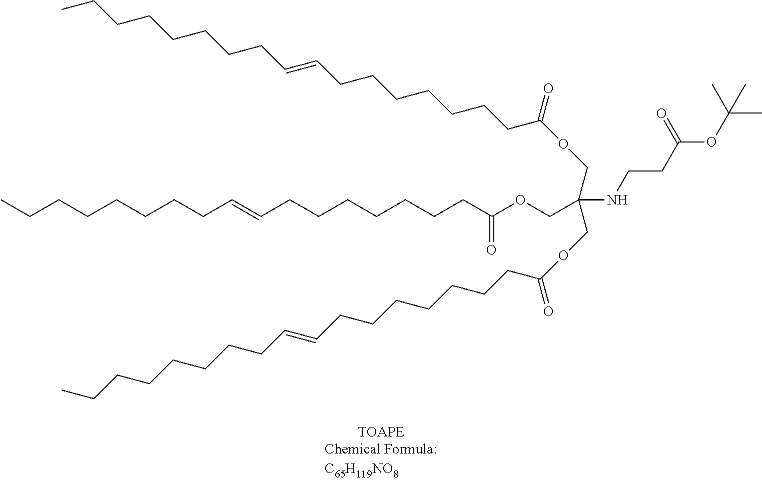 [SMILES: CCCCCCCC/C=C/CCCCCCCC(=O)OCC(COC(=O)CCCCCCC/C=C/CCCCCCCC)(COC(=O)CCCCCCC/C=C/CCCCCCCC)NCCC(=O)OC(C)(C)C]

### Tri-Linoleoyl Amino Propionic Acid Tert-Butyl Ester (TLAPE)

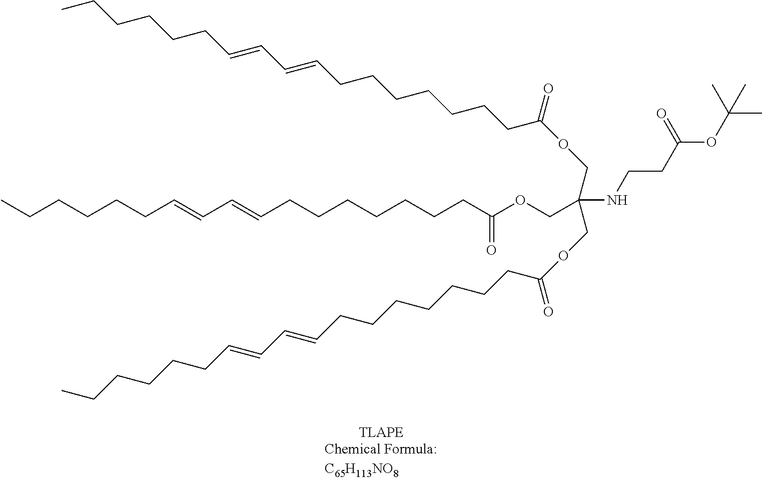 [SMILES: CCCCCC/C=C/C=C/CCCCCCCC(=O)OCC(COC(=O)CCCCCCC/C=C/C=C/CCCCCC)(COC(=O)CCCCCCC/C=C/C=C/CCCCCC)NCCC(=O)OC(C)(C)C]

### Tri-LinoLenoyl Amino Propionic Acid Tert-Butyl Ester (TLLAPE)

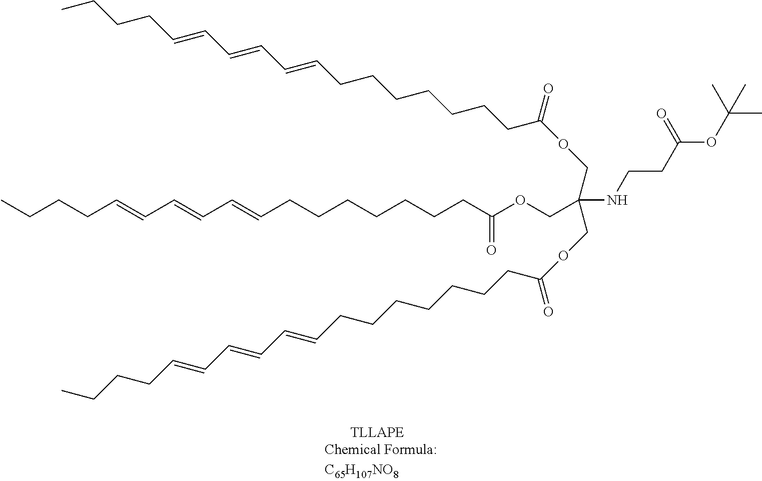 [SMILES: CCCC/C=C/C=C/C=C/CCCCCCCC(=O)OCC(COC(=O)CCCCCCC/C=C/C=C/C=C/CCCC)(COC(=O)CCCCCCC/C=C/C=C/C=C/CCCC)NCCC(=O)OC(C)(C)C]

The pH-responsive lipids of formula 2 were named with the following acronyms:

### Formula 2(a) I: Mono-Stearoyl Amino Propionic Acid (MSAPA)

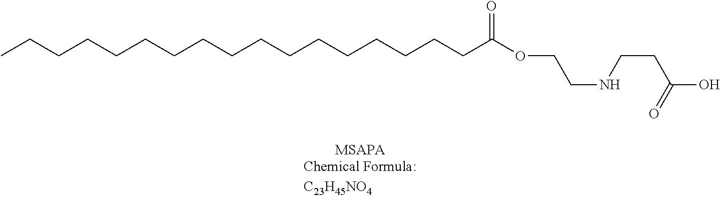 [SMILES: CCCCCCCCCCCCCCCCCC(=O)OCCNCCC(=O)O]

### Formula 2(a) II: Mono-Oleoyl Amino Propionic Acid (MOAPA)

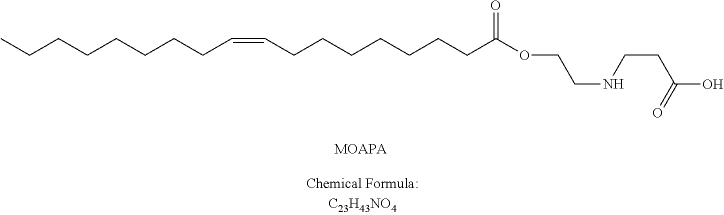 [SMILES: CCCCCCCC/C=C\CCCCCCCC(=O)OCCNCCC(=O)O]

### Formula 2(a) III: Mono-Linoleoyl Amino Propionic Acid (MLAPA)

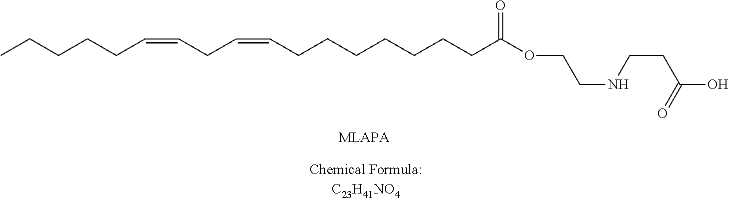 [SMILES: CCCCC/C=C\C/C=C\CCCCCCCC(=O)OCCNCCC(=O)O]

### Formula 2(a) IV: Mono-LinoLenoyl Amino Propionic Acid (MLLAPA)

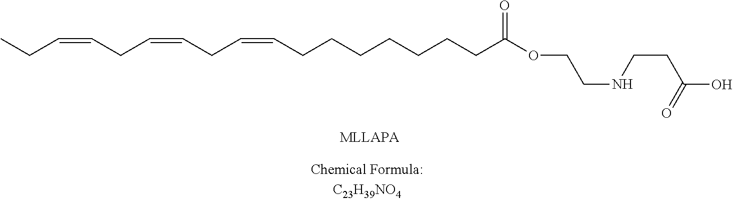 [SMILES: CC/C=C\C/C=C\C/C=C\CCCCCCCC(=O)OCCNCCC(=O)O]

### Formula 2(b) I: Di-Stearoyl Amino Propionic Acid (DSAPA)

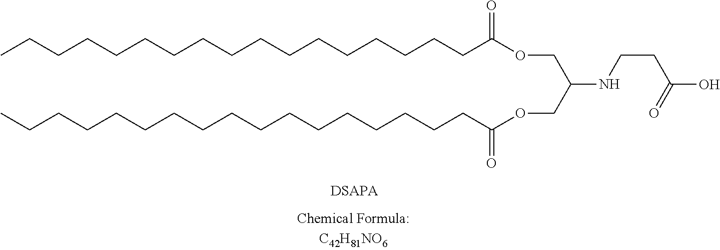 [SMILES: CCCCCCCCCCCCCCCCCC(=O)OCC(COC(=O)CCCCCCCCCCCCCCCCC)NCCC(=O)O]

### Formula 2(b) II: Di-Oleoyl Amino Propionic Acid (DOAPA)

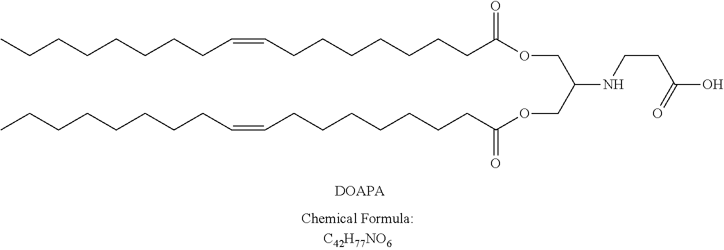 [SMILES: CCCCCCCC/C=C\CCCCCCCC(=O)OCC(COC(=O)CCCCCCC/C=C\CCCCCCCC)NCCC(=O)O]

### Formula 2(b) III: Di-Linoleoyl Amino Propionic Acid (DLAPA)

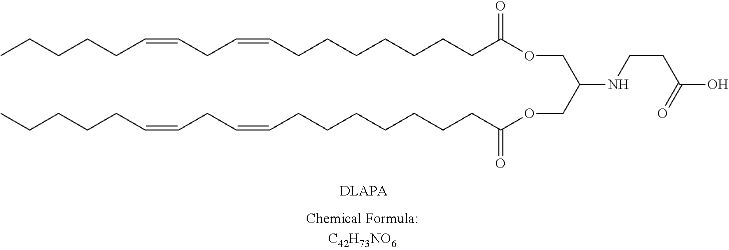 [SMILES: CCCCC/C=C\C/C=C\CCCCCCCC(=O)OCC(COC(=O)CCCCCCC/C=C\C/C=C\CCCCC)NCCC(=O)O]

### Formula 2(b) IV: Di-LinoLenoyl Amino Propionic Acid (DLLAPA)

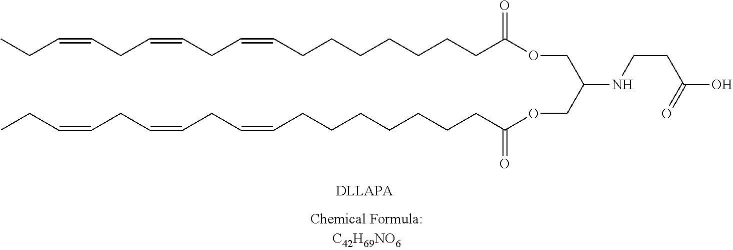 [SMILES: CC/C=C\C/C=C\C/C=C\CCCCCCCC(=O)OCC(COC(=O)CCCCCCC/C=C\C/C=C\C/C=C\CC)NCCC(=O)O]

### Formula 2(c) I: Tri-Stearoyl Amino Propionic Acid (TSAPA)

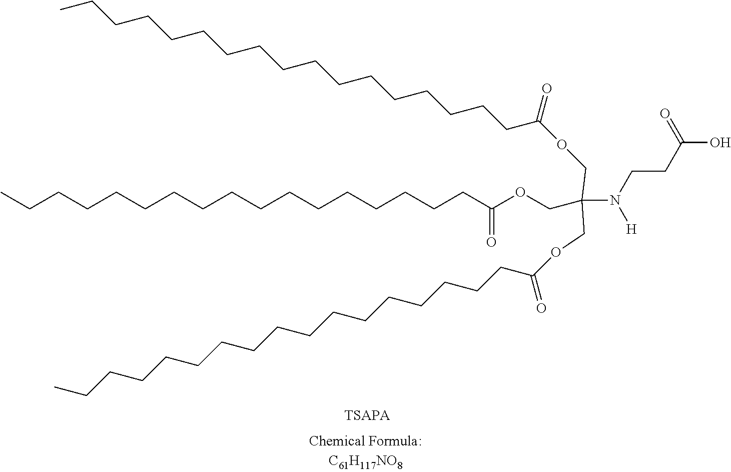 [SMILES: CCCCCCCCCCCCCCCCCC(=O)OCC(COC(=O)CCCCCCCCCCCCCCCCC)(COC(=O)CCCCCCCCCCCCCCCCC)NCCC(=O)O]

### Formula 2(c) II: Tri-Oleoyl Amino Propionic Acid (TOAPA)

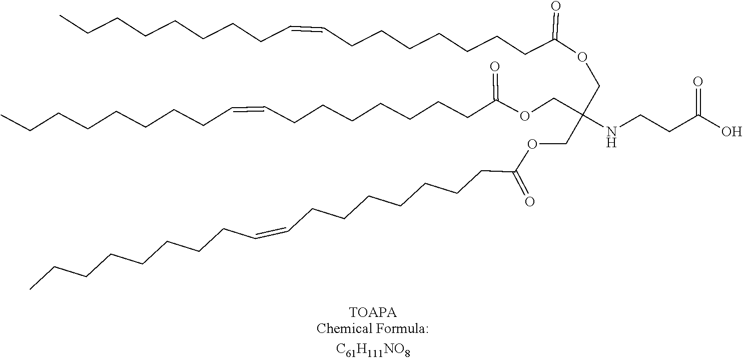 [SMILES: CCCCCCCC/C=C\CCCCCCCC(=O)OCC(COC(=O)CCCCCCC/C=C\CCCCCCCC)(COC(=O)CCCCCCC/C=C\CCCCCCCC)NCCC(=O)O]

### Formula 2(c) III: Tri-Linoleoyl Amino Propionic Acid (TLAPA)

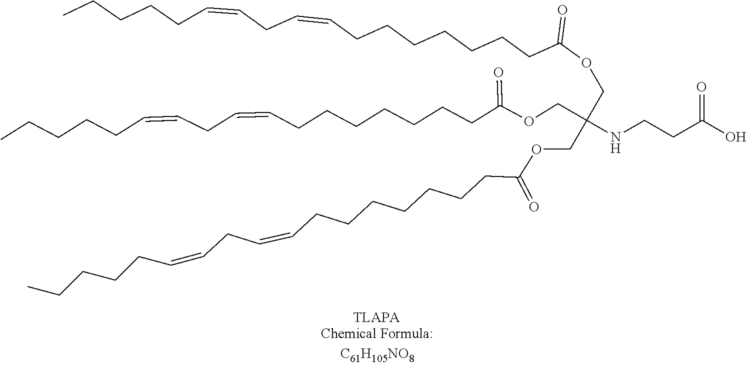 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCC(=O)O]

### Formula 2(c) IV: Tri-LinoLenoyl Amino Propionic Acid (TLLAPA)

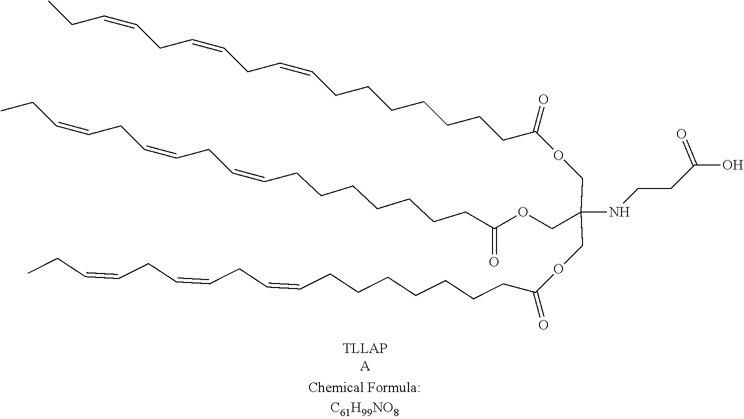 [SMILES: CC/C=C\C/C=C\C/C=C\CCCCCCCC(=O)OCC(COC(=O)CCCCCCC/C=C\C/C=C\C/C=C\CC)(COC(=O)CCCCCCC/C=C\C/C=C\C/C=C\CC)NCCC(=O)O]

The synthetic steps as depicted in schemes 1 to 3 above are described below in detail.

### General Procedure for Mono Michael Addition

To a solution of tert-butyl acrylate 2 in alcohol, an amine 1, 5 or 8 was added at room temperature and stirred for 4-30 h at 25 to 45° C. temperature. Alcohol and excess tert-butyl acrylate were evaporated in vacuo and the resulting residue was recrystallized or column purified using hexane and ethyl acetate (3:1) to yield mono Michael addition product (Compounds 3, 6 or 9 in schemes 1-3).

### Example 1

### Synthesis of tert-butyl 3-((2-hydroxyethyl)amino)propanoate (Compound 3)

To a solution of tert-butyl acrylate (compound 2) (1.05 mol) in methanol (500 mL), 2-aminoethanol (compound 1) (1.0 mol) was added at room temperature and stirred for 24 h at the same temperature. Methanol and excess tert-butyl acrylate were evaporated in vacuo and the resulting residue was purified by column chromatography using hexane and ethyl acetate (3:1) to yield compound 3 as a thick oil (80%).

### Example 2

### Synthesis of tert-butyl 3-((1,3-dihydroxypropan-2-yl)amino)propanoate (Compound 6)

To a solution of tert-butyl acrylate (Compound 2) (1.10 mol) in ethanol (300 mL), 2-amino-1,3-propanediol (compound 5) (1.0 mol) was added at room temperature and stirred for 4-5 h at the same temperature. Ethanol and excess tert-butyl acrylate were evaporated in vacuo and the resulting residue was recrystallized using hexane and ethyl acetate (3:1) to yield compound 6 as a white solid (92%).

### Example 3

### Synthesis of tert-butyl 3-((1, 3-dihydroxy-2-(hydroxymethyl)propan-2-yl)amino)propanoate (Compound 9)

To a solution of tert-butyl acrylate (compound 2) (1.0 mol) in ethanol (250 mL), Trizma (compound 8) (0.1 mol) was added at 45° C. and stirred for 30 h at the same temperature. Ethanol and excess tert-butyl acrylate were evaporated in vacuo and the resulting residue was recrystallized using hexane and ethyl acetate (3:1) to yield compound 9 as a white solid (90%).

### General Procedure (Esterification) for Synthesis of the Compound of Formula 1

Fatty acid was added to a stirred mixture of mono Michael adduct (compound 3, 6 or 9), DCC, and DMAP in dry DCM under a nitrogen atmosphere at room temperature (RT). The resulting reaction mixture was further stirred RT for 18-24 h. From the reaction mass, precipitated dicyclohexylurea was removed by filtration. The organic layer (filtrate) was evaporated under reduced pressure and obtained residue was purified by column chromatography (silica gel #70-230 and 10-15% ethyl acetate in hexane as eluent) to yield ester derivative of formula 1.

### Example 4

### Synthesis of 2-((3-(tert-butoxy)-3-oxopropyl)amino)ethyl stearate (MSAPE; 4 (I))

To a mixture of compound 3 (25 mmol), DCC (25 mmol) and DMAP (2.5 mmol) in dry DCM (30 mL) was added stearic acid (25.1 mmol) at RT, and stirred for 20 h. The product was isolated as white solid using general procedure (78%). The purified product was analysed by FT-IR, NMR (¹H and ¹³C) and HRMS.

### Example 5

### Synthesis of 2-((3-(tert-butoxy)-3-oxopropyl)amino)ethyl oleate (MOAPE; 4(II))

Following the procedure of example 4 except that the molar equivalent of oleic acid is substituted for stearic acid. Compound 4(II) was synthesized, isolated and purified as per the general procedure (81%). The purified product was analysed by FT-IR, NMR (¹H and ¹³C) and HRMS.

### Example 6

### Synthesis of 2-((3-(tert-butoxy)-3-oxopropyl)amino)ethyl (9Z,12Z)-octadeca-9,12-dienoate (MLAPE; 4(III))

Following the procedure of example 4 except that the molar equivalent of linoleic acid is substituted for stearic acid. Compound 4(III) was synthesized, isolated and purified as per the general procedure (80%). The purified product was analysed by FT-IR, NMR (¹H and ¹³C) and HRMS.

### Example 7

### Synthesis of 2-((3-(tert-butoxy)-3-oxopropyl)amino)ethyl (9Z,12Z,15Z)-octadeca-9,12,15-trienoate (MLLAPE; 4(IV))

Following the procedure of example 4 except that the molar equivalent of linolenic acid is substituted for stearic acid. 4(IV) was synthesized, isolated and purified as per the general procedure (74%). The purified product was analysed by FT-IR, NMR (¹H and ¹³C) and HRMS.

### Example 8

### Synthesis of 2-((3-(tert-butoxy)-3-oxopropyl)amino)propane-1,3-diyl distearate (DSAPE: 7(I))

To a mixture of compound 6 (25 mmol), DCC (50 mmol) and DMAP (2.5 mmol) in dry DCM (40 mL) was added stearic acid (50.25 mmol) at RT, and stirred for 24 h. The product was isolated as white solid using general procedure (81%). The purified product 7(I) was analysed by FT-IR, NMR (¹H and ¹³C) and HRMS.

### Example 9

### Synthesis of 2-((3-(tert-butoxy)-3-oxopropyl)amino)propane-1,3-diyl dioleate (DOAPE: 7(II))

Following the procedure of example 8 except that the molar equivalent of oleic acid is substituted for stearic acid. Compound 7(II) was synthesized, isolated and purified as per the general procedure (88%). The purified product was analysed by FT-IR, NMR (¹H and ¹³C) and HRMS.

### Example 10

### Synthesis of 2-((3-(tert-butoxy)-3-oxopropyl)amino)propane-1,3-diyl (9Z,9′Z,12Z,12′Z)-bis(octadeca-9,12-dienoate) (DLAPE: 7(III))

Following the procedure of example 8 except that the molar equivalent of linoleic acid is substituted for stearic acid. Compound 7(III) was synthesized, isolated and purified as per the general procedure (85%). The purified product was analysed by FT-IR, NMR (¹H and ¹³C) and HRMS.

### Example 11

### Synthesis of 2-((3-(tert-butoxy)-3-oxopropyl)amino)propane-1,3-diyl (9Z,9′Z,12Z,12′Z,15Z,15′Z)-bis(octadeca-9,12,15-trienoate) (DLLAPE: 7(IV))

Following the procedure of example 8 except that the molar equivalent of linolenic acid is substituted for stearic acid. Compound 7(IV) was synthesized, isolated and purified as per the general procedure (73%). The purified product was analysed by FT-IR, NMR (¹H and ¹³C) and HRMS.

### Example 12

### Synthesis of 2-((3-(tert-butoxy)-3-oxopropyl)amino)-2-((stearoyloxy)methyl) propane-1,3-diyl distearate. (TSAPE: 10(I))

To a mixture of compound 3 (4.01 mmol), DCC (12.83 mmol) and DMAP (2.0 mmol) in dry DCM (30 mL) was added stearic acid (12.43 mmol) at RT, and stirred for 22 h. The product was isolated as white solid using general procedure (78%). The purified product compound 10(I) was analysed by FT-IR, NMR (¹H and ¹³C) and HRMS.

### Example 13

### Synthesis of 2-((3-(tert-butoxy)-3-oxopropyl)amino)-2-((((Z)-octadec-9-enoyl)oxy) methyl)propane-1,3-diyl(9Z,9′Z)-bis(octadec-9-enoate) (TOAPE: 10(II))

Following the procedure of example 12 except that the molar equivalent of oleic acid is substituted for stearic acid. Compound 10(II) was synthesized, isolated and purified as per the general procedure (83%). The purified product was analysed by FT-IR, NMR (¹H and ¹³C) and HRMS.

### Example 14

### Synthesis of 2-((3-(tert-butoxy)-3-oxopropyl)amino)-2-((((9Z,12Z)-octadeca-9,12-dienoyl)oxy)methyl)propane-1,3-diyl (9Z,9′Z,12Z,12′Z)-bis(octadeca-9,12-dienoate) (TLAPE: 10(III))

Following the procedure of example 12 except that the molar equivalent of linoleic acid is substituted for stearic acid. Compound 10(III) was synthesized, isolated and purified as per the general procedure (80%). The purified product was analysed by FT-IR, NMR (¹H and ¹³C) and HRMS.

### Example 15

### Synthesis of 2-((3-(tert-butoxy)-3-oxopropyl)amino)-2-((((9Z,12Z,15Z)-octa-dec-9,12,15-trienoyl)oxy)methyl)propane-1,3-diyl(9Z,9′Z,12Z,12′Z,15Z,15′Z)-bis(octadeca-9,12,15-trienoate) (TLLAPE: 10(IV))

Following the procedure of example 12 except that the molar equivalent of linolenic acid is substituted for stearic acid. Compound 10(IV) was synthesized, isolated and purified as per the general procedure (70%). The purified product was analysed by FT-IR, NMR (¹H and ¹³C) and HRMS.

### General Procedure (Hydrolysis) for the Synthesis of the DH-Responsive Lipid of Formula 2

Tert-butyl ester derivative (formula 1) was added to a mixture of dry dichloromethane (DCM), trimethylamine (TFA) and triisopropylsilane (TIPS) (5:4:1 v/v/v) and resulting mixture was stirred at RT for 4-6 h. The solvent was removed in vacuo. Chloroform was added to the resulting residue and azeotropically distilled out to remove excess of TFA and TIPS. This stripping step was repeated two more times with chloroform to ensure complete removal of reagents. The obtained residue was purified by column chromatography (silica gel #70-230 and 10% methanol in chloroform as eluent) and vacuum dried for 48 h to obtain the final compound of formula 2.

### Example 16

### Synthesis of 3-((2-(stearoyloxy)ethyl)amino)propanoic acid (MSAPA)

Compound 4(I) (1 mmol) was added in a mixture of DCM (8.0 mL), TFA (6.4 mL) and TIPS (1.6 mL) and stirred at RT for 4 h. the compound of formula 2a(I) was isolated as a white semisolid (83%). The purified product was analysed by FT-IR, NMR (¹H and ¹³C) and HRMS

### Example 17

### Synthesis of 3-((2-(oleoyloxy)ethyl)amino)propanoic acid (MOAPA)

Following the procedure of example 16 except that the molar equivalent of compound 4 (II) is substituted for compound 4 (I). Formula 2a(II) was isolated as thick colourless oil (87%). The purified product was analysed by FT-IR, NMR (¹H and ¹³C) and HRMS.

### Example 18

### Synthesis of 3-((2-(((9Z,12Z)-octadeca-9,12-dienoyl)oxy)ethyl)amino)propanoic acid (MLAPA)

Following the procedure of example 16 except that the molar equivalent of compound 4 (III) is substituted for compound 4(I). Formula 2a(III) was isolated as yellowish thick oil (82%). The purified product was analysed by FT-IR, NMR (¹H and ¹³C) and HRMS.

### Example 19

### Synthesis of 3-((2-(((9Z,12Z,15Z)-octadeca-9,12,15-trienoyl)oxy)ethyl)amino) propanoic acid (MLLAPA)

Following the procedure of example 16 except that the molar equivalent of compound 4 (IV) is substituted for compound 4(I). Formula 2a(IV) was isolated as thick brown oil (77%). The purified product was analysed by FT-IR, NMR (¹H and ¹³C) and HRMS.

### Example 20

### Synthesis of 3-((1,3-bis(stearoyloxy)propan-2-yl)amino)propanoic acid (DSAPA)

Following the procedure of example 16 except that the molar equivalent of compound 7 (I) is substituted for compound 4(I). Formula 2b(I) was isolated as white solid (84%). The purified product was analysed by FT-IR, NMR (¹H and ¹³C) and HRMS.

### Example 21

### Synthesis of 3-((1,3-bis(oleoyloxy)propan-2-yl)amino)propanoic acid (DOAPA)

Following the procedure of example 16 except that the molar equivalent of compound 7 (II) is substituted for compound 4 (I). Formula 2b (II) was isolated as thick colourless oil (83%). The purified product was analysed by FT-IR, NMR (¹H and ¹³C) and HRMS.

### Example 22

### Synthesis of 3-((1,3-bis(((9Z,12Z)-octadeca-9,12-dienoyl)oxy)propan-2-yl)amino) propanoic acid (DLAPA)

Following the procedure of example 16 except that the molar equivalent of compound 7 (III) is substituted for compound 4(I). Formula 2b(III) was isolated as yellowish thick oil (83%). The purified product was analysed by FT-IR, NMR (¹H and ¹³C) and HRMS.

### Example 23

### Synthesis of 3-((1,3-bis(((9Z,12Z,15Z)-octadeca-9,12,15-trienoyl)oxy)propan-2-yl) amino)propanoic acid (DLLAPA)

Following the procedure of example 16 except that the molar equivalent of compound 7 (IV) is substituted for compound 4 (I). Formula 2b (IV) was isolated as brown thick oil (76%). The purified product was analysed by FT-IR, NMR (¹H and ¹³C) and HRMS.

### Example 24

### Synthesis of 3-((1,3-bis(stearoyloxy)-2-((stearoyloxy)methyl)propan-2-vi)amino) propanoic acid (TSAPA)

Following the procedure of example 16 except that the molar equivalent of compound 10 (I) is substituted for compound 4 (I). Formula 2c (I) was isolated as white solid (86%). The purified product was analysed by FT-IR, NMR (¹H and ¹³C) and HRMS.

### Example 25

### Synthesis of 3-((1,3-bis(((Z)-octadec-9-enoyl)oxy)-2-((((Z)-octadec-9-enoyl) oxy) methyl)propan-2-yl)amino)propanoic acid (TOAPA)

Following the procedure of example 16 except that the molar equivalent of compound 10 (II) is substituted for compound 4 (I). Formula 2c (II) was isolated a clear thick oil (82%). The purified product was analysed by FT-IR, NMR (¹H and ¹³C) and HRMS.

### Example 26

### Synthesis of 3-((1,3-bis(((9Z,12Z)-octadeca-9,12-dienoyl)oxy)-2-((((9Z,12Z)-octadeca-9,12-dienoyl)oxy)methyl)propan-2-yl)amino)propanoic acid (TLAPA)

Following the procedure of example 16 except that the molar equivalent of compound 10 (III) is substituted for compound 4(I). Formula 2c(III) was isolated a clear thick oil (80%). The purified product was analysed by FT-IR, NMR (¹H and ¹³C) and HRMS.

### Example 27

### Synthesis of 3-((1,3-bis(((9Z,12Z,15Z)-octadeca-9,12,15-trienoyl)oxy)-2-((((9Z,12Z, 15E)-octadeca-9,12,15-trienoyl)oxy)methyl)propan-2-yl)amino) propanoic acid (TLLAPA)

Following the procedure of example 16 except that the molar equivalent of compound 10 (IV) is substituted for compound 4(I). Formula 2c(IV) was isolated a yellowish thick oil (83%). The purified product was analysed by FT-IR, NMR (¹H and ¹³C) and HRMS.

### In-Vitro Cytotoxicity Study

The determination of non-toxic dosages of newly synthesized materials is critical for biomedical applications. As a result, Cytotoxicity studies were employed to determine the viability of cells after exposure to the synthesized ester derivatives (formula 1) and pH-responsive lipids (formula 2) of the invention. An in-vitro cell culture system using 3-(4,5-dimethylthiazol-2-yl)-2,5-diphenyltetrazolium bromide (MTT) assay was used to determine the biosafety of the formula 1 and formula 2 of the invention. Referring to FIG. 1, Graph A to X shows the cytotoxicity profile of the all synthesized lipids of the invention (formula 1 and formula 2) at various concentrations against human liver hepatocellular carcinoma (HepG2), human breast adenocarcinoma (MCF 7), and human cervix adenocarcinoma (HeLa). The percent cell survival for all ester intermediates and pH-responsive lipids was >75% against all the cell lines tested. No dose-dependent trends were observed in the percentage cell viability for any of the test materials, across all cell lines, within the concentration range studied. Test materials displaying cell viabilities greater than 75% can be considered to be of low toxicity and biologically safe. These findings confirmed their non-toxicity to mammalian cells and opened the further path for their use for drug delivery applications.

2. Formulation and Charactersation of Liposomes

The pH-responsive lipids of the invention are capable of forming disperse aqueous solutions of small bilayer structures (encapsulators) which can be employed to facilitate delivery of various molecules into a biological system, such as cells.

The invention, therefore, extends to methods for utilising the novel pH-responsive lipids of the invention to form pH-responsive encapsulants such as liposomes, as well as a composition comprising an encapsulator particle selected from the group consisting of liposomes, emulsions, micelles and lipidic bodies, wherein the encapsulator comprises the pH-responsive lipid of the current invention.

The following exemplary embodiment describes the preparation of pH-responsive liposomes using the novel pH-responsive lipids of the invention. The exemplary embodiment further describes the loading of the pH-responsive liposomes with an antibiotic, and the testing of the antibiotic loaded liposome.

In this embodiment of the invention, the antibiotic Vancomycin is used, however, it is envisaged that the pH-responsive liposomes could be loaded with any of a number of drugs, not just antibiotics, including, anticancer, anti-asthmatic small molecules, nucleosides, nucleotides, nucleic acids, polynucleotides, oligonucleotides, antibodies, toxins, negatively charged polymers and other polymers, for example proteins, peptides, hormones, carbohydrates, or polyamines.

### 2.1 Preparation of Liposomes

Thin film hydration, a robust, economic and broadly used technique was employed for the preparation of both conventional liposomes to act as controls and pH-responsive liposomes of the invention. Briefly, the pH-responsive lipids of the invention (formula 2), PC S100 and Chol (1:3:1; w/w/w) were dissolved in an appropriate quantity of chloroform in a round bottom flask (RBF). Subsequently, chloroform was evaporated at 40° C. by using a rotary evaporator until a thin lipid film was formed. The RBF with thin lipid film was then kept in a vacuum desiccator for overnight to remove trapped residual chloroform. The dried lipid film was then hydrated with 10 mL of Milli-Q water (10 mg/mL lipid concentration) for 4 hours at RT. These dispersions were vortexed for 2 minutes to remove any adhered lipid and then sonicated at 30% amplitude for 7 min. Non-responsive conventional liposomes were prepared by using the same method using PC S100 and Chol (mass ratio 1:3).

Preliminary optimization studies were conducted to determine the quantity of pH-responsive Lipid required for the formation of stable liposomes. Preliminary studies indicated that the magnitude of the surface charge of the liposomes increased with increasing concentration of pH-responsive Lipids (tested 5 to 40% w/w). This observation could be attributed to an increase in the number of ionizable hydrophilic head groups at vesicles surface. However, liposomes containing concentrations of pH-responsive Lipid greater than 20% w/w showed phase separation (vesicles aggregation) after an average storage period of 24-36 h. This instability can be the result of the increased number of pH-responsive lipid's hydrocarbon chains in the liposomes system disrupting the bilayer packing with Chol and PC tails. Such vesicles with disturbed bilayer packings have more gaps and are highly permeable to water and other small molecules.

Based on these observations, a 1:3:1 w/w ratio of pH-responsive lipid:PC:Chol was selected for the preparation of liposomes.

The optimized formulations (drug-free and drug loaded) prepared with this ratio were selected for further investigations.

The pH-responsive liposomes of the invention were subsequently loaded with Vancomycin by using 0.1% VCM solution (10 mL) as aqueous medium for lipid hydration.

pH-insensitive liposomes PC:Chol-VCM-Lipo were also loaded with Vancomycin as a control group.

### 2.2 Characterisation of Liposomes

### Dynamic Light Scattering

Mean vesicle diameter (MVD), polydispersity index (PDI) and zeta potential (ZP) measurements were performed using Malvern zeta sizer (Nano ZS Zetasizer, Malvern Instruments Corp, UK) working on the principle of photon correlation spectroscopy. The liposomal formulation was appropriately diluted with suitable PBS (pH 7.4, 6.5. 5.5 and 4.5) and then the measurements were performed at 2500. All the measurements were performed in triplicate.

TABLE 1
Particle size, PDI, zeta potential of
TSAPA-VCM-Lipo at different pH values.

| pH | Size (nm) | PDI | ZP (mV) |
|---|---|---|---|
| 4.5 | 149.33 ± 20.15 | 0.48 ± 0.11 | +4.55 ± 0.36 |
| 5.5 | 135.37 ± 18.80 | 0.47 ± 0.13 | −0.04 ± 0.29 |
| 6.5 | 112.28 ± 11.91 | 0.29 ± 0.05 | −3.70 ± 0.30 |
| 7.4 | 103.77 ± 00.93 | 0.21 ± 0.03 | −9.11 ± 0.56 |

TABLE 2
Particle size, PDI, zeta potential of
TOAPA-VCM-Lipo at different pH values

| pH | Size (nm) | PDI | ZP (mV) |
|---|---|---|---|
| 4.5 | 113.87 ± 3.13 | 0.27 ± 0.01 | 16.97 ± 1.03 |
| 5.5 | 108.80 ± 4.79 | 0.20 ± 0.01 | −01.11 ± 0.02 |
| 6.5 | 103.47 ± 5.13 | 0.16 ± 0.00 | −11.52 ± 1.26 |
| 7.4 | 105.60 ± 5.38 | 0.16 ± 0.01 | −23.77 ± 1.40 |

TABLE 3
Particle size, PDI, zeta potential of
TLAPA-VCM-Lipo at different pH values

| pH | Size (nm) | PDI | ZP (mV) |
|---|---|---|---|
| 4.5 | 126.15 ± 14.51 | 0.24 ± 0.07 | 16.63 ± 0.57 |
| 5.5 | 118.55 ± 16.82 | 0.26 ± 0.05 | −01.71 ± 0.36 |
| 6.5 | 100.77 ± 4.22 | 0.19 ± 0.01 | −10.83 ± 0.83 |
| 7.4 | 103.69 ± 4.46 | 0.19 ± 0.01 | −22.00 ± 3.80 |

TABLE 4
Particle size, PDI, zeta potential of
TLLAPA-VCM-Lipo at different pH values

| pH | Size (nm) | PDI | ZP (mV) |
|---|---|---|---|
| 4.5 | 119.75 ± 11.61 | 0.23 ± 0.04 | 14.87 ± 0.75 |
| 5.5 | 114.47 ± 11.61 | 0.23 ± 0.04 | −2.07 ± 0.07 |
| 6.5 | 104.30 ± 17.96 | 0.26 ± 0.11 | −13.10 ± 1.67 |
| 7.4 | 99.38 ± 6.59 | 0.22 ± 0.05 | −23.73 ± 4.67 |

TABLE 5
Particle size, PDI, zeta potential of
DSAPA-Lipo at different pH values

| pH | Size (nm) | PDI | ZP (mV) |
|---|---|---|---|
| 4.5 | 104.6 ± 0.92 | 0.25 ± 0.06 | 8.42 ± 1.17 |
| 5.5 | 125.93 ± 7.68 | 0.30 ± 0.07 | 4.46 ± 0.34 |
| 6.5 | 106.57 ± 2.00 | 0.23 ± 0.00 | −3.56 ± 0.67 |
| 7.4 | 104.03 ± 2.58 | 0.21 ± 0.01 | −12.2 ± 1.23 |

TABLE 6
Particle size, PDI, zeta potential of
DOAPA-Lipo at different pH values

| pH | Size (nm) | PDI | ZP (mV) |
|---|---|---|---|
| 4.5 | 98.21 ± 1.52 | 0.24 ± 0.01 | 14.6 ± 1.82 |
| 5.5 | 105.50 ± 2.01 | 0.22 ± 0.01 | 4.38 ± 1.40 |
| 6.5 | 104.17 ± 2.15 | 0.25 ± 0.01 | −2.02 ± 0.88 |
| 7.4 | 101.12 ± 1.17 | 0.21 ± 0.01 | −11.00 ± 0.26 |

TABLE 7
Particle size, PDI, zeta potential of
DLAPA-Lipo at different pH values

| pH | Size (nm) | PDI | ZP (mV) |
|---|---|---|---|
| 4.5 | 102.87 ± 2.81 | 0.21 ± 0.02 | 13.83 ± 1.89 |
| 5.5 | 104.17 ± 3.41 | 0.21 ± 0.02 | 5.93 ± 1.48 |
| 6.5 | 116.93 ± 3.23 | 0.30 ± 0.02 | −1.87 ± 0.87 |
| 7.4 | 101.47 ± 2.04 | 0.22 ± 0.01 | −10.62 ± 3.09 |

TABLE 8
Particle size, PDI, zeta potential of
DLLAPA-Lipo at different pH values

| pH | Size (nm) | PDI | ZP (mV) |
|---|---|---|---|
| 4.5 | 103.00 ± 1.42 | 0.20 ± 0.00 | 9.76 ± 2.17 |
| 5.5 | 103.80 ± 0.71 | 0.19 ± 0.00 | 5.10 ± 1.40 |
| 6.5 | 106.50 ± 0.68 | 0.21 ± 0.01 | −3.15 ± 0.93 |
| 7.4 | 101.30 ± 1.10 | 0.20 ± 0.01 | −10.2 ± 3.11 |

TABLE 9
Particle size, PDI, zeta potential of
MSAPA-Lipo at different pH values

| pH | Size (nm) | PDI | ZP (mV) |
|---|---|---|---|
| 4.5 | 109.00 ± 1.70 | 0.28 ± 0.01 | 01.33 ± 0.28 |
| 5.5 | 111.00 ± 2.50 | 0.23 ± 0.01 | 00.34 ± 0.41 |
| 6.5 | 109.10 ± 2.49 | 0.22 ± 0.01 | −03.00 ± 1.23 |
| 7.4 | 105.00 ± 9.07 | 0.21 ± 0.09 | −10.60 ± 1.20 |

TABLE 10
Particle size, PDI, zeta potential of
MOAPA-Lipo at different pH values

| pH | Size (nm) | PDI | ZP (mV) |
|---|---|---|---|
| 4.5 | 142.01 ± 28.90 | 0.26 ± 0.05 | 06.39 ± 0.45 |
| 5.5 | 138.02 ± 17.62 | 0.25 ± 0.04 | 00.34 ± 0.41 |
| 6.5 | 137.31 ± 21.93 | 0.24 ± 0.05 | −09.12 ± 3.67 |
| 7.4 | 133.52 ± 24.70 | 0.24 ± 0.03 | −15.02 ± 0.89 |

TABLE 11
Particle size, PDI, zeta potential of
MLAPA-Lipo at different pH values

| pH | Size (nm) | PDI | ZP (mV) |
|---|---|---|---|
| 4.5 | 191.11 ± 67.71 | 0.22 ± 0.04 | 02.44 ± 0.73 |
| 5.5 | 183.12 ± 49.53 | 0.22 ± 0.05 | −03.83 ± 1.33 |
| 6.5 | 167.20 ± 45.29 | 0.22 ± 0.02 | −14.80 ± 2.71 |
| 7.4 | 158.10 ± 31.22 | 0.28 ± 0.05 | −20.11 ± 5.33 |

TABLE 12
Particle size, PDI, zeta potential of
MLLAPA-Lipo at different pH values

| pH | Size (nm) | PDI | ZP (mV) |
|---|---|---|---|
| 4.5 | 168.60 ± 57.71 | 0.30 ± 0.01 | 06.39 ± 0.45 |
| 5.5 | 162.22 ± 48.67 | 0.20 ± 0.06 | 00.34 ± 0.41 |
| 6.5 | 159.62 ± 39.14 | 0.19 ± 0.01 | −09.12 ± 3.67 |
| 7.4 | 141.81 ± 51.90 | 0.20 ± 0.01 | −15.02 ± 0.89 |

TABLE 13
Particle size, PDI, zeta potential of
PC:Chol-Lipo at different pH values

| pH | Size (nm) | PDI | ZP (mV) |
|---|---|---|---|
| 4.5 | 275.00 ± 6.87 | 0.51 ± 0.02 | 0.21 ± 0.09 |
| 5.5 | 202.10 ± 5.97 | 0.39 ± 0.01 | 0.13 ± 0.10 |
| 6.5 | 211.30 ± 8.67 | 058 ± 0.10 | −1.80 ± 0.40 |
| 7.4 | 206.60 ± 4.59 | 0.51 ± 0.10 | −1.95 ± 0.29 |

### Effect of DH on Mean Vesicle Diameters and Polydispersity Index

The Mean Vesicle Diameters for all VCM loaded liposomes (TSAPA-VCM-Lipo, TOAPA-VCM-Lipo, TLAPA-VCM-Lipo TLLAPA-VCM-Lipo and PC:Chol-VCM-Lipo) and blank liposomes (DSAPA-Lipo, DOAPA-Lipo, DLAPA-Lipo, DLLAPA-Lipo, MSAPA-Lipo, MOAPA-Lipo, MLAPA-Lipo and MLLAPA-Lipo) at different pH values are presented in Tables 1 to 13 above.

Effect of DH on zeta potential

The surface charge-switching behaviour of all the liposomal formulations was confirmed by determining their ZP values at different pH values. As presented in FIGS. 2, 3 and 4 and in table 13 above the zeta potential of the pH-insensitive PC:Chol-VCM-Lipo system remained unchanged (approximately zero) regardless of pH value. On the other hand, as presented in FIGS. 2, 3 and 4 and in Tables 1 to 12 above, a significant change (p<0.0008) in the zeta potential of all the liposome formulations consisting of pH-responsive Lipids occurred upon a change in pH. The trend observed was a shift of ZP to more positive side with a decrease in the pH. These results confirmed the surface charge-switching behaviour imparted by PH-responsive lipid to the liposomes.

The change in ZP can be ascribed to the protonation/deprotonation mechanism and presence of a free carboxylic acid function in the pH-responsive lipids structure. At physiological pH of 7.4, the secondary amine was neutral and in this situation ZP value was predominantly due to the free carboxylic acid while as the pH was lowered, the protonation of secondary amine occurred and this has increased the intensity of positive charge leading to shifting of ZP to more positive side. Thus, the pH-responsive lipids are capable of giving pH-switchable behaviour to the liposomes confirmed by a change in the zeta potential according to the surrounding pH. This is a characteristic behaviour of zwitterionic/pH-responsive lipids including those with carboxylic acid and an amine group. Based on these results, it can be concluded that in the liposomal structure, the propionic acid group of pH-responsive lipid extended towards the aqueous phase, a secondary amine contiguous to the bilayer interface and hydrocarbon chain(s) into the bilayer.

### Transmission Electron Microscopy (TEM)

Referring to FIG. 5, the morphology of VCM loaded liposomes was examined using TEM instrument (Jeol, JEM-1010, Japan). Briefly, a diluted liposomal sample (2 μl) was placed on 3 mM form an (0.5% plastic powder in amyl acetate) coated copper grid (300 mesh), allowed to dry, stained with 2% uranyl acetate for one min and visualized using a TEM at an accelerating voltage of 100 kV.

The negative stain images revealed nanometric sized particles and showed a homogeneous population of vesicles. They confirmed the presence of well identified unilamellar spherical particles with a large internal space. Images obtained by TEM are in agreement with the results obtained by dynamic light scattering (DLS) spectrophotometry.

### Entrapment Efficiency (% EE) and Drug Loading (DL)

Entrapment efficiency was determined as the percentage of VCM encapsulated into the liposomes by the centrifugal-ultrafiltration method. Briefly, an aliquot (2 mL) of the liposome sample was placed in the upper chamber of the ultrafiltration centrifugal tube (Amicon® Ultra-4, Centrifugal Filter Units, Millipore, USA, MWCO=10 kDa) and was centrifuged for 30 min at 3500 rpm at 25° C. The ultrafiltrate was appropriately diluted with Milli-Q water and unentrapped drug concentration was determined by UV-visible spectrophotometer (UV-1650 PC, Shimadzu, Japan) at 280.0 nm. The regression equation and coefficient were y=0.0045x−0.0019 and 0.9999 respectively. All the experiments were performed in triplicate.

The entrapment efficiency (% EE) and drug loading capacity (% DL) were calculated by using following equations.

% EE=(W_{TD}−W_{FD})/W_{TD}×100

Where W_{TD} is total drug in the liposomal formulation and W_{FD} is total free drug in the filtrate obtained after ultrafiltration.

% DL=W_{ED}/W_{T}×100

Where, W_{ED} is the weight of drug entrapped and W_{T} is the total weight of entrapped drug, PC, Chol, and PH-RESPONSIVE LIPID.

The % EE for TSAPA-VCM-Lipo, TOAPA-VCM-Lipo, TLAPA-VCM-Lipo, and TLLAPA-VCM-Lipo was 39.74±1.06, 44.85±5.94, 29.93±1.90 and 29.14±1.63 respectively whereas % DL was 4.04±0.25, 4.65±1.24, 2.86±0.66 and 2.80±0.32 respectively (Table 14). The liposomes without pH-responsive lipids had % EE and DL of 37.83±2.57 and 2.39±0.12 respectively which is considered consistent with the known literature reported values.

TABLE 14
Encapsulation efficiency (EE) and drug loading
capacity (DLC) of VCM loaded liposomes.

| Liposomes | EE (%) | DL (%) |
|---|---|---|
| PC:Chol-VCM-Lipo | 37.83 ± 2.57 | 2.39 ± 0.12 |
| TSAPA-VCM-Lipo | 39.74 ± 1.06 | 4.04 ± 0.25 |
| TOAPA-VCM-Lipo | 44.85 ± 5.94 | 4.65 ± 1.24 |
| TLAPA-VCM-Lipo | 29.93 ± 1.90 | 2.86 ± 0.66 |
| TLLAPA-VCM-Lipo | 29.14 ± 1.66 | 2.803 ± 0.322 |

### In-Vitro Drug Release

The present invention also relates to the delivery of drugs to cells. In exemplary embodiments, the invention relates to lipidic nano and/or micro drug delivery systems. Referring to FIG. 10, the mechanism by which the pH-responsive liposomes operate as a targeted drug delivery system is explained.

In-vitro drug release of VCM from the pH-responsive liposomes of the invention is illustrated in FIG. 6. At all-time intervals, VCM release at acidic pH (6.5) was higher than at physiological pH of 7.4. After 8 hours, the percentage cumulative VCM release at pH 7.4 from TSAPA-VCM-Lipo, TOAPA-VCM-Lipo, TLAPA-VCM-Lipo and TLLAPA-VCM-Lipo was 42.48±5.01, 50.86±4.22, 53.76±5.60 and 57.30±4.73% respectively whereas at pH 6.5 it was 62.72±7.96, 71.64±0.55, 76.51±0.91 and 81.92±7.25% respectively.

The data clearly indicates that VCM release at pH 6.5 was 40-45% more than at pH 7.4 at the end of 8 hours. Although the release at pH 6.5 was faster than at pH 7.4, it was in a controlled manner over a period of 48 hours which shows that the developed pH-responsive liposomes of the invention are an ideal antibiotic delivery system. The percentage VCM released from the conventional liposomes (PC:Chol-VCM-Lipo) was more than 90% after 8 hours at both the studied pHs (7.4 and 6.5). All the drug was released from the control group of non-responsive liposomal systems within 24 h and it was pH independent.

For further confirmation, the mean dissolution time (MDT _{90%}) for 90% of drug release was calculated from in-vitro release data. The calculated MDT values for VCM release at pH 6.5 from all the pH-responsive liposomal formulations of the current invention were found to be lower than the MDT values at pH 7.4, as is shown in Table 15. The MDT value is inversely proportional to the release rate, i.e. lower the MDT higher the release rate and vice versa. Thus the obtained MDT values suggest that the drug release rate at acidic condition (pH 6.5) is faster than the release rate at physiological pH (7.4). The in-vitro release data and calculated MDT values, therefore, collectively suggest that the VCM release from all the liposomal formulations follow a sustained and pH-dependent release pattern. The higher drug release rate at the acidic environment from liposomes can be attributed to the alteration in the lipid bilayer orientation and permeability caused by conformational changes at the head group and hydrophobic tails of the PH-responsive lipid at low pH.

TABLE 15
Mean dissolution time (hours) calculated for 90%
of VCM release from liposomes at different pH

| | MDT_{90%} | | | | |
|---|---|---|---|---|---|
| | | TSAPA- | TOAPA- | TLAPA- | TLLAPA- |
| pH | VCM | Lipo | Lipo | Lipo | Lipo |
| 5.5 | 6.064 | 8.658 | 8.532 | 7.986 | 6.773 |
| 6.5 | 4.173 | 7.164 | 8.467 | 6.096 | 7.898 |
| 7.4 | 2.690 | 12.197 | 12.069 | 13.721 | 12.703 |

In-vitro drug release data also suggested that the release rate increases concurrently with an increase in the number of olefinic bonds (n) in PH-RESPONSIVE LIPID's hydrophobic chains. This trend was observed at both the studied pH values of 7.4 and 6.5. The percentage VCM release order at both the pH values was TSAPA-VCM-Lipo (n=0)<TOAPA-VCM-Lipo (n=1)<TLAPA-VCM-Lipo (n=2)<TLLAPA-VCM-Lipo (n=3). Low leakage of VCM from liposomes prepared using saturated PH-RESPONSIVE LIPID, could be due to the compact arrangement of saturated lipophilic chains in the vesicle bilayer. However, unsaturated PH-responsive lipid containing liposomes displayed more payload release. This could be due to a kink produced by an unsaturation in the lipid's hydrocarbon chain, which disrupts the regular periodic bilayer structure. This disruption increases more gaps in the bilayer which leads to increased permeability.

### In-Vitro Antibacterial Activity

The synthesized novel class of PH-responsive lipid of the invention were found to be good formulation ingredients to develop responsive nanosystems for antibiotics with enhanced and sustained in vitro activity at acidic conditions that exist at an infection site.

The minimum inhibitory concentration (MIC) values for VCM loaded liposomes were determined against Staphylococcus aureus (SA) and Methicillin-resistant Staphylococcus aureus (MRSA) using broth dilution method (Materials Science and Engineering: C, Volume 61, 2016, Pages 616-630). The stock bacterial cultures were grown in Mueller-Hinton Broth (MHB) 24 h before the test at 37° C.

Bacterial suspensions were prepared equal to 1.5×10⁸ CFU ml⁻¹ as half mcFarland. Serial dilutions of VCM loaded liposomal formulations were prepared in MHB broth then added to the bacterial culture, mixed properly and incubated at 37° C. for 24 hours. Thereafter, 10 μl was spotted on Mueller Hinton Agar (MHA) plates and incubated for 24 hours at 37° C. to determine the MIC values. The study continued for four days to determine the sustained release activity of encapsulated VCM. Experiments were performed in triplicate and drug-free liposomes, bare VCM solution and VCM loaded liposomes without Ph-responsive lipid were used as controls.

All the MICs (μg/mL) for bare VCM, VCM loaded responsive liposomes and VCM loaded conventional control liposome against S. aureus and MRSA at pH 7.4 and pH 6.5 are given in Table 16 and 17 respectively.

At the end of 24 h period MICs for bare VCM, TSAPA-VCM-Lipo, TOAPA-VCM-Lipo, TLAPA-VCM-Lipo and TLLAPA-VCM-Lipo at pH 7.4 were 8.79, 15.63, 14.32, 11.72, 11.72 and 0.98, 2.93, 3.91, 5.86, 5.86 against S. aureus and MRSA respectively and at pH 6.5 these values were 1.95, 1.95, 19.5, 3.42, 1.95 and 0.98, 0.98, 1.30, 1.96, 1.63 against S. aureus and MRSA respectively. Although VCM was most potent after 24 hours, it had no antibacterial activity thereafter. Contrarily, all the responsive liposomal formulations exhibited sustained antibacterial activity up to 96 h at both pH values (Table 4 and 5). This proves the superiority of nano antibiotic systems over bare antibiotics. At pH 6.5, the MIC values for responsive liposomes were almost 5.75-fold and 3.16-fold lower against S. aureus and MRSA respectively than at pH 7.4. The lower MICs at acidic pH were justifiable according to the drug release pattern from liposomes. The release of VCM at pH 6.5 was low at pH 7.4 and almost 40-45% more at pH 6.5. Hence, the decrease in the MIC's value (increased potency). The MICs obtained for responsive liposomes were lower than those obtained for previously reported surface charge-switchable polymeric nanoparticles. For comparison, the MIC values for PC:Chol-VCM liposomes were determined against S. aureus and MRSA at pH 6.5 and 7.4 to check the pH-dependent enhancement in antibacterial activity of prepared responsive liposomes. The MICs for this non-responsive liposomal system after 24 h were 2.93, 1.95 against S. aureus and 1.93 and 11.72 against MRSA at pH 7.4 and pH 6.5 respectively. These values were pH independent (no lowering of MIC at acidic pH) and were comparable with MICs observed for free VCM. Further, no activity was exhibited by PC:Chol-VCM liposomes after 48 h. These results supported the finding that pH-responsive liposomes had greater antibacterial potential (low MICs) with sustained activity at acidic pH.

TABLE 16
Minimum inhibitory concentration (MIC) values at pH 7.4

| | | MIC (μg/mL) | | | | | | | |
|---|---|---|---|---|---|---|---|---|---|
| | | DAY - I | | DAY - II | | DAY - III | | DAY - IV | |
| Entry | Liposomes | S. aureus | MRSA | S. aureus | MRSA | S. aureus | MRSA | S. aureus | MRSA |
| 1 | Bare VCM | 3.91 | 7.81 | NA | NA | NA | NA | NA | NA |
| 2 | TSAPA-VCM-Lipo | 1.95 | 2.93 | 16.60 | 3.91 | 312.50 | 2.93 | 500.0 | 2.93 |
| 3 | TOAPA- VCM-Lipo | 14.32 | 5.86 | 16.28 | 4.56 | 187.50 | 22.79 | 312.5 | 500.0 |
| 4 | TLAPA-VCM-Lipo | 11.72 | 6.51 | 16.93 | 10.42 | 11.72 | 7.81 | NA | 19.53 |
| 5 | TLLAPA- VCM-Lipo | 11.72 | 8.14 | 28.65 | 6.84 | 63.80 | 7.16 | 187.5 | 255.2 |
| 6 | PC:CHOL- VCM-Lipo | 2.93 | 2.93 | NA | 16.60 | NA | 4.88 | NA | NA |

TABLE 17
Minimum inhibitory concentration (MIC) values at pH 6.5

| | | MIC (μg/mL) | | | | | | | |
|---|---|---|---|---|---|---|---|---|---|
| | | DAY - I | | DAY - II | | DAY - III | | DAY - IV | |
| Entry | Liposomes | S. aureus | MRSA | S. aureus | MRSA | S. aureus | MRSA | S. aureus | MRSA |
| 1 | Bare VCM | 7.81 | 15.62 | NA | NA | NA | NA | NA | NA |
| 2 | TSAPA-VCM-Lipo | 1.95 | 11.72 | 1.95 | 2.93 | 78.13 | 16.11 | NA | 16.11 |
| 3 | TOAPA- VCM-Lipo | 1.95 | 0.98 | 3.91 | 5.86 | 2.93 | 1.95 | 2.93 | 1.46 |
| 4 | TLAPA- VCM-Lipo | 3.91 | 2.44 | 0.98 | 2.44 | 2.93 | 4.88 | 2.93 | 4.39 |
| 5 | TLLAPA- VCM-Lipo | 2.44 | 1.46 | 0.98 | 2.44 | 2.93 | 2.93 | 0.98 | 2.44 |
| 6 | PC:CHOL- VCM-Lipo | 1.95 | 11.72 | NA | NA | NA | NA | NA | NA |

### Stability

Referring to FIGS. 8A and 8B, The physical appearance, MVD, PDI and ZP of VCM loaded liposomes were evaluated for 3 months at 4° C. and RT. All the liposomal formulations were stable at 4° C. for the period of 3 months, as indicated by no particle aggregation, change in colour, and no significant difference in MVD, PDI and ZP.

### Animals:

All the experiments performed using animals were approved by Animal Research Ethics committee of the University of KwaZulu-Natal. In vivo study, the protocol was approved by Animal Research Ethics committee of the University of KwaZulu-Natal. The protocol approval numbers for in vivo skin infection model and transdermal permeation studies were AREC/104/015PD and AREC/054/14/Animal respectively. BALB/c mice and Wistar rats were used for in vivo skin infection model and transdermal permeation study respectively. Animals used in the study were procured from Biomedical Resource Unit of University of KwaZulu-Natal, Westville, Durban, South Africa.

### In-Vivo Antibacterial Activity

The only formulations which were most active in vitro antibacterial testing were evaluated further for in vivo antibacterial activity to restrict the uses of a number of animals. In-vivo skin infection studies on BALB/c mice proved the potential of TOAPA-VCM-Lipo and TLAPA-VCM-Lipo as effective nano antibiotics. Referring to FIG. 7, it can be seen that there was a significant reduction (p<0.0001) of bacterial loads in the skin lesions treated with formulation compared to VCM only or no treatment. The mean bacterial load (number of CFU) recovered from non-treated skin wound was 2.94±0.25 log₁₀ CFU per skin lesion which was almost 4.4- and 14.7-fold higher than that found in TOAPA-VCM-Lipo and TLAPA-VCM-Lipo treated mice respectively. Isolated bacterial load (log₁₀ CFU) from treated skin wounds with TOAPA-VCM-Lipo and TLAPA-VCM-Lipo were 0.67±0.51 and 0.210±0.15 respectively.

### In Vitro Transdermal Permeation Study

All the intermediate, ester derivatives (MSAPE, MOAPE, MLAPE, MLLAPE, DSAPE, DOAPE, DLAPE, DLLAPE, TSAPE, TOAPE, TLAPE and TLLAPE) were evaluated as potential CPEs for transdermal drug delivery using tenofovir (TNF) as a model drug. The experiments were performed on shaved rat skin using Franz diffusion cells. TNF gels were prepared using hydroxypropyl methyl cellulose, milli-Q water and 1% w/w of compounds of formula 1. The permeability of TNF in the absence of enhancers was performed as a control. The experiments revealed that at the end of six hours, the cumulative amount of TNF that permeated through the skin without CPE (compounds of formula 1) was 253.10±23.84 μg cm⁻² (Table 18). TNF was able to permeate the skin without any permeation enhancer with a steady state flux value 040.91±04.93 μg cm⁻² h⁻¹ (Table 6).

Referring to FIG. 9, the results indicated that the all the ester derivatives (compounds of formula 1) studied enhanced the permeability of TNF across the skin (Table 6). Among all the tested derivatives MOAPE, DLAPE, MLLAPE, MLAPE, TLAPE and TSAPE significantly increased the steady-state flux (Jss) of TNF with enhancement ratio (ER) of 5.58, 3.95, 3.70, 2.25, 2.11 and 2.09 respectively (Table 6). These experiments proved the potential of compounds of formula 1 as promising CPEs for the delivery of bioactives.

TABLE 18
Effect of the various derivatives on
the transdermal permeability of TNF

| Enhancer | Cumulative | Jss | Permeability | |
|---|---|---|---|---|
| (1% w/w) | amount | (flux) | (P × 10⁻²) | ER |
| Control | 253.1 ± 23.84 | 040.91 ± 04.93 | 0.204 ± 0.02 | 1.00 |
| MOAPE | 1594.06 ± 198.92 | 228.40 ± 33.45 | 1.142 ± 0.16 | 5.58 |
| MSAPE | 407.12 ± 66.51 | 063.48 ± 12.41 | 0.317 ± 0.06 | 1.55 |
| MLAPE | 574.97 ± 74.09 | 092.07 ± 13.72 | 0.460 ± 0.06 | 2.25 |
| MLLAPE | 945.88 ± 44.93 | 151.58 ± 08.04 | 0.758 ± 0.04 | 3.70 |
| DOAPE | 424.95 ± 59.84 | 070.07 ± 10.89 | 0.350 ± 0.05 | 1.71 |
| DSAPE | 336.1 ± 25.67 | 057.33 ± 05.64 | 0.286 ± 0.02 | 1.40 |
| DLAPE | 1074.62 ± 61.12 | 161.87 ± 11.32 | 0.809 ± 0.05 | 3.95 |
| DLLAPE | 400.37 ± 18.92 | 067.58 ± 02.53 | 0.337 ± 0.01 | 1.65 |
| TOAPE | 342.3 ± 26.94 | 061.39 ± 04.52 | 0.306 ± 0.02 | 1.50 |
| TSAPE | 531.70 ± 23.87 | 085.76 ± 03.69 | 0.428 ± 0.02 | 2.09 |
| TLAPE | 574.39 ± 88.70 | 086.47 ± 11.54 | 0.432 ± 0.05 | 2.11 |
| TLLAPE | 453.53 ± 37.43 | 077.80 ± 08.07 | 0.389 ± 0.04 | 1.79 |

1. A synthesized ester intermediate of formula 1. 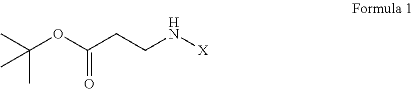
Wherein 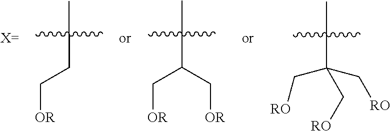
And wherein R is a saturated or unsaturated fatty acid (C12-C20).

2. The synthesised ester intermediate as claimed in claim 1 characterised in that the ester intermediate comprises a hydrophilic head group, functionalized with beta-amino propionic acid (beta alanine) tert butyl ester and connected to 1, 2 or 3 fatty acid chains through an acid-labile ester bond or linker.

3. The synthesised ester intermediate as claimed in claim 2 characterised in that the linker comprises any of 2-aminoethanol (ethanolamine) (HO(CH₂)₂NH₂), 2-amino-1,3-propanediol (serinol) ((HOCH₂)₂CHNH₂), or 2-amino-2-(hydroxymethyl) propane-1,3-diol (trizma or Trisaminomethane) ((HOCH₂)₃CNH₂).

4. The synthesised ester intermediate of any of claims 1 to 3, characterised in that R is any of C18H36O2 (stearic acid), C18H34O2 (oleic acid), C18H32O2 (linoleic acid) or C18H30O2 (linolenic acid).

5. The synthesised ester intermediate as claimed in any of claims 1 to 4, characterised in that the ester intermediate comprises one or more or the following: 2-((3-(tert-butoxy)-3-oxopropyl)amino)ethyl stearate (MSAPE); 2-((3-(tert-butoxy)-3-oxopropyl)amino)ethyl oleate (MOAPE); 2-((3-(tert-butoxy)-3-oxopropyl)amino)ethyl (9Z,12Z)-octadeca-9,12-dienoate (MLAPE); 2-((3-(tert-butoxy)-3-oxopropyl)amino)ethyl (9Z,12Z,15Z)-octadeca-9,12,15-trienoate (MLLAPE); 2-((3-(tert-butoxy)-3-oxopropyl)amino)propane-1,3-diyl distearate (DSAPE); 2-((3-(tert-butoxy)-3-oxopropyl)amino)propane-1,3-diyl dioleate (DOAPE); 2-((3-(tert-butoxy)-3-oxopropyl)amino)propane-1,3-diyl (9Z,9′Z,12Z,12′Z)-bis(octadeca-9,12-dienoate) (DLAPE); 2-((3-(tert-butoxy)-3-oxopropyl)amino)propane-1,3-diyl (9Z,9′Z,12Z,12′Z,15Z,15′Z)-bis(octadeca-9,12,15-trienoate) (DLLAPE); 2-((3-(tert-butoxy)-3-oxopropyl)amino)-2-((stearoyloxy)methyl) propane-1,3-diyl distearate, (TSAPE); 2-((3-(tert-butoxy)-3-oxopropyl)amino)-2-((((Z)-octadec-9-enoyl)oxy)methyl)propane-1,3-diyl(9Z,9′Z)-bis(octadec-9-enoate) (TOAPE); 2-((3-(tert-butoxy)-3-oxopropyl)amino)-2-((((9Z,12Z)-octadeca-9,12-dienoyl)oxy)methyl)propane-1,3-diyl (9Z,9′Z,12Z,12′Z)-bis(octadeca-9,12-dienoate); 2-((3-(tert-butoxy)-3-oxopropyl)amino)-2-((((9Z,12Z,15Z)-octa-dec-9,12,15-trienoyl)oxy)methyl propane-1,3-diyl(9Z,9′Z,12Z,12′Z,15Z,15′Z)-bis(octadeca-9,12,15-trienoate) (TLLAPE).

6. The synthesised ester intermediate as claimed in any of claims 1 to 5 in which the terminal ester group of the compound of formula 1 is hydrolysed to create a pH-responsive lipid of formula 2 (a, b or c): 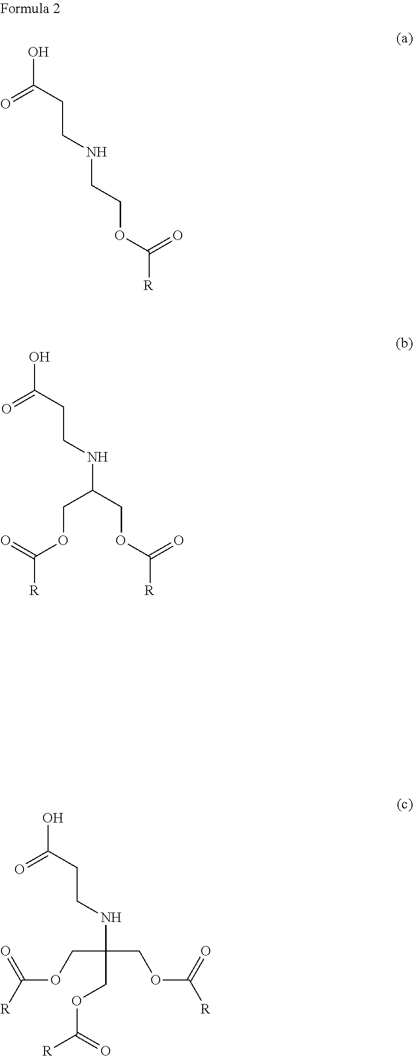
where R is a saturated or unsaturated fatty acid chain (C12-C20).

7. The pH-responsive lipid of formula 2, as claimed in claim 6, in which the synthesised pH-responsive lipid comprises a hydrophilic head group, functionalized with beta-amino propionic acid (beta alanine) and connected to 1, 2 or 3 fatty acid chains through an acid-labile ester bond.

8. The pH-responsive lipid of formula 2 as claimed in either of claim 6 or 7 in which R is selected from C₁₈H₃₆O₂ (stearic acid), C₁₈H₃₄O₂ (oleic acid), C₁₈H₃₂O₂ (linoleic acid) or C₁₈H₃₀O₂ (linolenic acid).

9. The pH-responsive lipid of formula 2 as claimed in claim 8 in which the pH-responsive lipid comprises any of the following:
2(a):3-((2-(stearoyloxy)ethyl)amino) propanoic acid (MSAPA); 3-((2-(oleoyloxy)ethyl) amino)propanoic acid (MOAPA); 3-((2-(((9Z,12Z)-octadeca-9,12-dienoyl)oxy)ethyl)amino) propanoic acid (MLAPA); 3-((2-(((9Z,12Z,15Z)-octadeca-9,12,15-trienoyl)oxy)ethyl)amino) propanoic acid (MLLAPA);
2(b): 3-((1,3-bis(stearoyloxy)propan-2-yl)amino)propanoic acid (DSAPA); 3-((1,3-bis (oleoyloxy)propan-2-yl)amino)propanoic acid (DOAPA); 3-((1,3-bis(((9Z,12Z)-octadeca-9,12-dienoyl)oxy)propan-2-yl)amino)propanoic acid (DLAPA); 3-((1,3-bis(((9Z,12Z,15Z)-octadeca-9,12,15-trienoyl)oxy)propan-2-yl)amino)propanoic acid (DLLAPA); or
2(c): 3-((1,3-bis(stearoyloxy)-2-((stearoyloxy)methyl)propan-2-yl)amino) propanoic acid (TSAPA); or 3-((1,3-bis(((Z)-octadec-9-enoyl)oxy)-2-((((Z)-octadec-9-enoyl)oxy)methyl propan-2-yl) amino)propanoic acid (TOAPA); or 3-((1,3-bis(((9Z,12Z)-octadeca-9,12-dienoyl)oxy)-2-((((9Z,12Z)-octadeca-9,12-dienoyl)oxy) methyl)propan-2-yl)amino) propanoic acid (TLAPA) or 3-((1,3-bis(((9Z,12Z,15Z)-octadeca-9,12,15-trienoyl)oxy)-2-((((9Z,12Z,15Z)-octadeca-9,12,15-trienoyl)oxy) methyl)propan-2-yl)amino) propanoic acid (TLLAPA).

10. A method of synthesising the pH-responsive lipid of formula 2 as claimed in any one of claims 6 to 9 and containing a secondary amine group, the method comprising a selective mono Michael addition reaction in between amino group of any of ethanolamine or serinol or trizma with tert-butyl acrylate at specific reaction conditions.

11. The pH-responsive lipid as claimed in any one of claims 6 to 9 for use in the delivery of bioactive pharmaceutical agents, including but not limited to small molecules, lipids, nucleosides, nucleotides, nucleic acids, polynucleotides, oligonucleotides, antibodies, toxins, negatively charged polymers and other polymers, for example proteins, peptides, hormones, carbohydrates, or polyamines across cellular membranes.

12. The pH-responsive lipid as claimed in any one of claims 6 to 9 for use in a nanosystem in which the nanosystem includes but is not limited to a liposome.

13. A liposome as claimed in claim 12 in which the liposome comprises the pH-responsive lipid of the invention and one or more additional lipid compounds.

14. A liposome as claimed in claim 13 in which the liposome comprises between 5 and 40 w/w % of said pH-responsive lipid of formula 2.

15. A liposome as claimed in claim 13 in which the liposome comprises between 5 and 20 w/w % of said pH-responsive lipid of formula 2.

16. A liposome as claimed in any one of claims 13 to 15 in which the additional lipid compounds include any of cholesterol, phosphatidylcholine (PC), phosphatidyl ethanolamine, ceramide, sphingolipid, tetraether lipid, or diacylglycerol, phosphatidylserine, phosphatidic acid or CHEMS.

17. A liposome as claimed in claim 16 in which the liposome comprises a combination of pH-responsive lipid, phosphatidylcholine and cholesterol.

18. A liposome as claimed in claim 17 in which the ratio of pH-responsive lipid:phosphatidylcholine:cholesterol is 1:3:1 (w/w/w).

19. A liposome as claimed in any of claims 13 to 18 in which the liposome has an average size of between 80 to 600 nm.

20. A liposome as claimed in any of claims 13 to 18 in which the liposome additionally comprises a medically active substance including, but not limited to drugs molecules, peptides nucleosides, nucleotides, nucleic acids, polynucleotides, oligonucleotides, antibodies, and toxins.

21. The use of the pH-responsive liposomes as claimed in any one of claims 13 to 20 as a pH-responsive nano drug delivery system for site-specific drug delivery.

22. The synthesised ester intermediates of formula 1 as claimed in any of claims 1 to 5 for use as chemical permeation enhancers for drug delivery applications.

23. The synthesised ester intermediates of formula 1 as claimed in any of claims 1 to 5 for use in the transdermal delivery of bioactive pharmaceutical agents, including but not limited to small molecules, lipids, nucleosides, nucleotides, nucleic acids, polynucleotides, oligonucleotides, antibodies, toxins, negatively charged polymers and other polymers, for example proteins, peptides, hormones, carbohydrates, or polyamines.
